(19) United States
(12) Patent Application Publication
(10) Pub. No.: US 2026/0185131 A1
(43) Pub. Date: Jul. 2, 2026

(54) BETA-KETOACYL-ACP SYNTHASE IV VARIANTS

(71) Applicant: Corbion Biotech, Inc., South San Francisco, CA (US)

(72) Inventors: Joshua FERREIRA, South San Francisco, CA (US); Janice Lau WEE, South San Francisco, CA (US); Nien-Hsi KO, South San Francisco, CA (US)

(73) Assignee: Corbion Biotech, Inc., South San Francisco, CA (US)

(21) Appl. No.: 19/542,551

(22) Filed: Feb. 17, 2026

Related U.S. Application Data
(63) Continuation of application No. 17/793,123, now Pat. No. 12,600,994.
(60) Provisional application No. 62/961,996, filed on Jan. 16, 2020.

(51) Int. Cl. C12P 7/6445; C12N 9/10; C12N 9/16; C12N 15/62; C12N 15/74

(52) U.S. Cl. CPC C12P 7/6445; C12N 9/1029; C12N 9/16; C12N 15/62; C12N 15/74; C12Y 203/01041

(57) ABSTRACT

Provided are non-natural or variant β-ketoacyl-acyl carrier protein (ACP) synthase (KAS) IVa enzymes (KASIVa), polynucleotides encoding such variant KASIVa, host cells expressing such variant KASIVa, oils and oil products produced by such cells, and methods of making and using such variant KASIVa.

### CROSS REFERENCE TO RELATED APPLICATION

This patent application is the continuation of U.S. patent application Ser. No. 17/793,123, filed on Jul. 15, 2022, which is the national stage of PCT Application No. PCT/US2021/013575, filed on Jan. 15, 2021, which claims the benefit of U.S. Provisional Patent Application No. 62/961,996, filed Jan. 16, 2020, each of which is incorporated by reference in its entirety herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 99,640 Bytes XML file named “138515-0601_SL.xml,” dated Feb. 13, 2026.

### BACKGROUND

Prototheca moriformis base or wild-type strains produce oils with primarily palmitic acid (C16:0) and oleic acid (C18:1), which account for nearly 85% of the fatty acids present at the end of fermentation. The amount of medium-chain fatty acids, C8:0 to C14:0, is negligible with C12:0 and C14:0 present at low, non-zero levels. Chain lengthening from short chain fatty acids to longer chain fatty acids is catalyzed by β-ketoacyl-ACP synthase (KAS) enzymes. Medium-chain fatty acid levels near zero imply that the endogenous KAS enzymes in P. moriformis are not evolved for producing medium-chain fatty acids.

### SUMMARY

In one aspect, provided are polynucleotides encoding a non-natural or variant β-ketoacyl-ACP synthase (KAS) IVa enzyme (KASIVa). In some embodiments, the non-natural KASIVa comprises at least about 80%, 85%, 90%, 91%, 92%, 93%, 94%, 95%, 96%, 97%, 98% or 99% identity to amino acid residues 34-523 of SEQ ID NO: 4 and comprises an X at the position corresponding to position 146; wherein X is an amino acid residue selected from the group consisting of glycine (G), asparagine (N), or serine (S), wherein the positions are with reference to SEQ ID NO: 4. In some embodiments, the non-natural KASIVa catalyzes the elongation of a medium-chain fatty acyl-ACP, e.g., from C8 to C10. In some embodiments, the non-natural KASIVa preferentially produces C10-ACP. In some embodiments, the non-natural KASIVa facilitates the production of increased levels of C10 fatty acids or the production of an oil with increased levels of C10 fatty acids in comparison to a wild-type KASIVa. In some embodiments, the non-natural KASIVa catalyzes the production of increased levels of C10 fatty acids in comparison to a wild-type KASIVa. In some embodiments, the non-natural KASIVa has increased activity on a C8-acyl substrate and/or has increased specificity for a C8-acyl substrate in the formation of the C10-acyl product (e.g., increases the ratio of C10 fatty acids to the sum of other fatty acids produced) in comparison to a wild-type KASIVa. In some embodiments, the X at position 146 is a serine (S) residue. In some embodiments, the X at position 146 is a glycine (G) residue. In some embodiments, the X at position 146 is asparagine (N). In some embodiments, the non-natural or variant KASIVa comprises a plastid transit peptide. In some embodiments, the plastid transit peptide comprises at least about 80%, 85%, 90%, 91%, 92%, 93%, 94%, 95%, 96%, 97%, 98% or 99% identity to amino acid residues 1-33 of SEQ ID NO: 3, SEQ ID NO: 4, SEQ ID NO: 10 or SEQ ID NO: 11. In some embodiments, the plastid transit peptide is encoded by a polynucleotide comprising at least about 80%, 85%, 90%, 91%, 92%, 93%, 94%, 95%, 96%, 97%, 98% or 99% identity to SEQ ID NO: 12. In some embodiments, the polynucleotide comprises at least about 80%, 85%, 90%, 91%, 92%, 93%, 94%, 95%, 96%, 97%, 98% or 99% identity to nucleic acid residues 100-1563 of SEQ ID NO: 7, SEQ ID NO: 9, or SEQ ID NO: 14. In some embodiments, the polynucleotide comprises at least about 80%, 85%, 90%, 91%, 92%, 93%, 94%, 95%, 96%, 97%, 98% or 99% identity to SEQ ID NO: 7, SEQ ID NO: 9, or SEQ ID NO: 14. In some embodiments, the polynucleotide comprises codon bias for improved expression in a microalgal host cell, e.g., a Prototheca or Chlorella microalgal host cell.

In another aspect, provided are expression cassettes comprising a polynucleotide encoding a non-natural or variant β-ketoacyl-ACP synthase (KAS) IVa enzyme (KASIVa), as described above and herein. In another aspect, provided are vectors comprising a polynucleotide encoding a non-natural or variant β-ketoacyl-ACP synthase (KAS) IVa enzyme (KASIVa), as described above and herein or an expression cassette comprising such a polynucleotide. In some embodiments, the vector further comprises a polynucleotide encoding an exogenous lipid biosynthesis enzyme, e.g., fatty acid biosynthesis enzymes and/or triglyceride biosynthesis enzymes. In some embodiments, the encoded thioesterase preferentially hydrolyzes C10-ACP substrates. In some embodiments, the thioesterase is a Cuphea FATB thioesterase. In some embodiments, the thioesterase is a Cuphea FATB thioesterase selected from the group consisting of Cuphea hookeriana FATB2 (ChFATB2), Cuphea paucipetala FATB1 (Cpau FATB1), Cuphea palustris FATB1 (Cpal FATB1), Cuphea ignea FATB1 (Cignea FATB1), Cuphea avigera FATB1 (Ca FATB1, Cuphea painteri FATB1 (Cpai FATB1), Cuphea crassiflora FATB1 (CcrasFATB1), Cuphea koehneana FATB3 (CkoeFATB3), Cuphea leptopoda FATB1 (CleptFATB1), Cuphea angustifolia FATB1 (CangFATB1), Cuphea llavea FATB1 (CllaFATB1), and Cuphea lophostoma FATB1 (ClopFATB1). In some embodiments, the encoded thioesterase comprises at least about at least about 80%, 85%, 90%, 91%, 92%, 93%, 94%, 95%, 96%, 97%, 98% or 99% identity to amino acid residues 39-392 of SEQ ID NO: 5, wherein the thioesterase catalyzes the production of increased levels of C10 fatty acids and/or has increased specificity for C10 fatty acids in comparison to a wild-type thioesterase.

In a further aspect, provided are non-natural or variant KASIVa polypeptides encoded by the polynucleotides, as described above and herein. In some embodiments, the KASIVa comprises an amino acid sequence of SEQ ID NO: 4, comprising an X at the position corresponding to position 146; wherein X is an amino acid residue selected from the group consisting of glycine (G), asparagine (N), or serine (S). In some embodiments, X is a serine (S) residue. In some embodiments, X is a glycine (G) residue. In some embodiments, X is a asparagine (N) residue. In a further aspect, provided are fusion proteins comprising the non-natural or variant KASIVa as described above and herein, and a heterologous or an exogenous peptide or polypeptide.

In a further aspect, provided are host cells comprising the polynucleotide encoding a non-natural or variant β-ketoacyl-ACP synthase (KAS) IVa enzyme (KASIVa), as described above and herein, an expression cassette and/or a vector comprising such a polynucleotide. In some embodiments, the host cell further comprises a polynucleotide encoding an exogenous lipid biosynthesis enzyme, e.g., a fatty acyl-ACP thioesterase. In some embodiments, the thioesterase preferentially hydrolyzes C10-ACP substrates. In some embodiments, the thioesterase is a Cuphea FATB thioesterase. In some embodiments, the thioesterase is a Cuphea FATB thioesterase selected from the group consisting of Cuphea hookeriana FATB2 (ChFATB2), Cuphea paucipetala FATB1 (Cpau FATB1), Cuphea palustris FATB1 (Cpal FATB1), Cuphea ignea FATB1 (Cignea FATB1), Cuphea avigera FATB1 (Ca FATB1, Cuphea painteri FATB1 (Cpai FATB1), Cuphea procumbens FATB1 (CprocFATB1), Cuphea procumbens FATB3 (CprocFATB3), Cuphea crassiflora FATB1 (CcrasFATB1), Cuphea koehneana FATB3 (CkoeFATB3), Cuphea leptopoda FATB1 (CleptFATB1), Cuphea angustifolia FATB1 (CangFATB1), Cuphea llavea FATB1 (CllaFATB1), and Cuphea lophostoma FATB1 (ClopFATB1), Cuphea PSR23 FatB3 (CuPSR23FATB3), Cuphea viscosissima FatB1 (CvisFATB1), and Cuphea glossostoma FatB1 (CgFATB1). In some embodiments, the thioesterase comprises at least about at least about 80%, 85%, 90%, 91%, 92%, 93%, 94%, 95%, 96%, 97%, 98% or 99% identity to amino acid residues 39-392 of SEQ ID NO: 5, wherein the thioesterase has increased activity on a C10-acyl substrate and/or has increased specificity for a C10-acyl substrate (e.g., increases the ratio of C10 fatty acids to the sum of other fatty acids produced) in comparison to a wild-type thioesterase. In some embodiments, one or more endogenous lipid biosynthesis enzymes (e.g., fatty acid biosynthesis enzymes and/or triglyceride biosynthesis enzymes) are selected from the group consisting of a fatty acyl thioesterase A (FATA), a fatty acyl thioesterase B (FATB), a 1-acylglycerol-3-phosphate O-acyltransferase (LPAAT), a glycerol-3-phosphate acyltransferase (GPAT), an acyl CoA:diacylglycerol acyltransferase (DGAT), a fatty acid elongase (FAE) and a long-chain acyl-CoA synthetase (LACS) are deleted, knocked-out or knocked down. In some embodiments, the host cells further comprise one or more exogenous or heterologous lipid biosynthesis enzymes selected from the group consisting of a fatty acyl thioesterase A (FATA), a fatty acyl thioesterase B (FATB), a 1-acylglycerol-3-phosphate O-acyltransferase (LPAAT), a glycerol-3-phosphate acyltransferase (GPAT), an acyl CoA:diacylglycerol acyltransferase (DGAT), and a fatty acid elongase (FAE), a long-chain acyl-CoA synthetase (LACS). In some embodiments, the host cell further comprises one or more exogenous or heterologous enzymes, such as a sucrose invertase and a 4-amino-5-hydroxymethyl-2-methylpyrimidine phosphate synthase (THIC). In some embodiments, the host cell is an oleaginous microbial cell (e.g., oleaginous yeasts, such as Yarrowia lipolytica). In some embodiments, the microbial host cell is an oleaginous microalgal cell. In some embodiments, host cell is a heterotrophic microalga, e.g., a obligate heterotrophic microalga. In some embodiments, the host cell is a microalga of the phylum Chlorpophya, e.g., of the class Trebouxiophytae, e.g., of the order Chlorellales, e.g., of the family Chlorellacae, e.g., of the genus Prototheca or Chlorella, e.g., of a species selected from the group consisting of Prototheca moriformis, Prototheca krugani, Prototheca stagnora, Prototheca zopfii and Chlorella protothecoides. In some embodiments, the host cell has a fatty acid profile comprising at least 50%, 55%, 60%, 65%, 70%, 75%, 80%, 85%, 90%, or more, C10 fatty acids.

In a further aspect, provided are methods of producing a host cell that produces an oil having a desired fatty acid profile. In some embodiments, the methods comprise transforming a microalgal host cell with a polynucleotide encoding a non-natural or variant β-ketoacyl-ACP synthase (KAS) IVa enzyme (KASIVa), as described above and herein, an expression cassette and/or a vector comprising such a polynucleotide, and cultivating the microalgal host cell so as to produce the oil. In some embodiments, the microalgal host cell produces on oil comprising at least 50%, 55%, 60%, 65%, 70%, 75%, 80%, 85%, 90%, or more, C10 fatty acids. In some embodiments, the microalgal host cell produces an oil with an increase C10 fatty acid level of at least 5%, 10%, 20%, 40%, 50%, 80%, 100%, 200%, or more, in comparison to an untransformed microalga or a microalga transformed with a wild-type acyl-ACP KASIVa. In some embodiments, the microalgal host cell produces an oil with an increase in C10 fatty acid level of at least 2-fold, 3-fold, 4-fold, 5-fold, 6-fold, 7-fold, 8-fold, 9-fold, 10-fold, 11-fold, 12-fold, 13-fold, 14-fold, 15-fold, or more, in comparison to an untransformed microalga or a microalga transformed with a wild-type acyl-ACP KASIVa. In some embodiments, the oil is a triglyceride oil. In some embodiments, the methods further comprise the step of recovering the oil.

In another aspect, provided are methods of producing an oil comprising predominantly C10 fatty acids. In some embodiments, the methods comprise transforming a host cell with polynucleotide encoding a non-natural or variant β-ketoacyl-ACP synthase (KAS) IVa enzyme (KASIVa), as described above and herein, an expression cassette and/or a vector comprising such a polynucleotide, and cultivating the microalgal host cell so as to produce an oil comprising at least about 50% C10 fatty acids, e.g., at least about 55%, 60%, 65%, 70%, 75%, 80%, 85%, 90%, or more, C10 fatty acids.

In another aspect, provided are methods for increasing the level of C10 fatty acids and/or the ratio of C10 fatty acids to the sum of other fatty acids composing the fatty acid profile of an oil produced by a host cell. In some embodiments, the methods comprise providing a parent gene encoding a KASIVa enzyme, mutating the gene so as to encode a non-natural or variant KASIVa as described above and herein; expressing the mutated gene in the host cell; and producing the oil, whereby the level of C10 fatty acids and/or the ratio of C10 fatty acids to the sum of other fatty acids composing the fatty acid profile of the oil are increased.

With respect to the methods, in some embodiments, the methods comprise co-expressing a polynucleotide encoding an exogenous lipid biosynthesis enzyme, e.g., a fatty acyl-ACP thioesterase. In some embodiments, the thioesterase preferentially hydrolyzes C10-ACP substrates. In some embodiments, the thioesterase is a Cuphea FATB thioesterase. In some embodiments, the thioesterase is a Cuphea FATB thioesterase selected from the group consisting of Cuphea hookeriana FATB2 (ChFATB2), Cuphea paucipetala FATB1 (Cpau FATB1), Cuphea palustris FATB1 (Cpal FATB1), Cuphea ignea FATB1 (Cignea FATB1), Cuphea avigera FATB1 (Ca FATB1, Cuphea painteri FATB1 (Cpai FATB1), Cuphea procumbens FATB1 (CprocFATB1), Cuphea procumbens FATB3 (CprocFATB3), Cuphea crassiflora FATB1 (CcrasFATB1), Cuphea koehneana FATB3 (CkoeFATB3), Cuphea leptopoda FATB1 (CleptFATB1), Cuphea angustifolia FATB1 (CangFATB1), Cuphea llavea FATB1 (CllaFATB1), and Cuphea lophostoma FATB1 (ClopFATB1), Cuphea PSR23 FatB3 (CuPSR23FATB3), Cuphea viscosissima FatB1 (CvisFATB1), and Cuphea glossostoma FatB1 (CgFATB1). In some embodiments, the thioesterase comprises at least about at least about 80%, 85%, 90%, 91%, 92%, 93%, 94%, 95%, 96%, 97%, 98% or 99% identity to amino acid residues 39-392 of SEQ ID NO: 5.

With respect to the methods, in some embodiments, the host cell is an oleaginous microbial cell (e.g., oleaginous yeasts, such as Yarrowia lipolytica). In some embodiments, the microbial host cell is an oleaginous microalgal cell. In some embodiments, host cell is a heterotrophic microalga, e.g., a obligate heterotrophic microalga. In some embodiments, the host cell is a microalga of the phylum Chlorpophya, e.g., of the class Trebouxiophytae, e.g., of the order Chlorellales, e.g., of the family Chlorellacae, e.g., of the genus Prototheca or Chlorella, e.g., of a species selected from the group consisting of Prototheca moriformis, Prototheca krugani, Prototheca stagnora, Prototheca zopfii and Chlorella protothecoides.

In a further aspect, provided is an oil produced by the methods described above and herein. With respect to the oil, in some embodiments, the oil comprises triglycerides, wherein the oil comprises at least about 35%, at least about 40%, at least about 41%, at least 42%, at least about 43%, or at least about 44% tridecanoin. In some embodiments, the oil is a microbial. In some embodiments, the oil comprises about 35% to about 55% of tridecanoin, about 40% to about 50% of tridecanoin, or about 42% to about 47% tridecanoin. In some embodiments, the oil comprises at least about 40%, at least about 45%, at least about 46%, at least about 47%, at least about 48%, at least about 49%, at least about 50%, or at least about 51%, of medium- and long-chain triglyceride (MLCT). In some embodiments, the oil further comprises about 40% to about 60% of medium- and long-chain triglyceride (MLLCT), or about 45% to about 55% of MLCT. In some embodiments, about at least about 5% of MLCT, at least about 10% of MLCT, optionally about 10% to about 15% of MLCT, in the oil is caprate-caprate-laurate (CaCaLa) triglyceride. In some embodiments, at least about 5% of MLCT, at least about 10% of MLCT, optionally about 10% to about 15% of MLCT, is caprate-oleate-caprate (CaOCa) triglyceride. In some embodiments, at least about 5% of MLCT, optionally about 5% to about 10% of MLCT, is caprate-caprate-palmitate (CaCaP) triglyceride. In some embodiments at least about 3%, at least about 5%, or optionally about 3% to about 8% of MLCT, is a combination of caprate-caprate-myristate (CaCaM) triglyceride and laurate-laurate-caprate (LaLaCa) triglyceride.

In some embodiments, the C10:0 fatty acid content of the oil is at least about 45%, at least about 50%, at least about 55%, at least about 60%, at least about 65%, or at least about 70% of total fatty acids derivable from the oil. In some embodiments, the C10:0 fatty acid content of the oil is about 40% to about 90%, about 50% to about 80%, about 60% to about 75%, or about 65% to about 75%, or about 70% to about 75% of total fatty acids derivable from the oil. In some embodiments, the C8:0 fatty acid content of the oil is present in an amount less than about 5%, less than about 4%, less than about 3%, less than about 2%, or less than 1% of total fatty acids derivable from the oil. In some embodiments, the C12:0 fatty acid content of the oil is present in an amount less than 15%, less than 10%, optionally about 2% to about 10%, of the total fatty acids derivable from the oil. In some embodiments, the C14:0 fatty acid content of the oil is present in an amount less than about 15%, less than about 10%, optionally about 2% to about 10%, of the total fatty acids derivable from the oil. In some embodiments, the oil is obtained from an oleaginous microbial cell. In some embodiments, the oil is obtained from an oleaginous microalga. In some embodiments, the oil further comprises ergosterol. In some embodiments, the oil further comprises additional sterols, wherein the most abundant sterol is ergosterol. In some embodiments, the oil further comprises brassicasterol. In some embodiments, the ratio of ergosterol to brassicasterol in the oil is at least 5:1, 10:1, 15:1 or 20:1. In some embodiments, the oil is a noninteresterified cell oil.

In a further aspect, provided is a product comprising the microbial oil and/or a chemical composition derived from the microbial oil disclosed herein.

### Definitions

As used herein, an “acyl-ACP thioesterase,” “fatty acyl-ACP thioesterase,” “acyl-ACP TE,” or “thioesterase.” interchangeably refer to an enzyme that catalyzes the cleavage of a fatty acid from an acyl carrier protein (ACP) during lipid synthesis. Acyl-acyl carrier protein (ACP) thioesterases (TEs) hydrolyze acyl-ACP thioester bonds, releasing free fatty acids and ACP.

The term “acyl-ACP preferring TE” refers to the fatty acyl-ACP substrate specificity of a TE. An acyl-ACP preferring TE preferentially liberates a particular fatty acid from an acyl-ACP substrate. For example, the acyl-ACP preferring TE can preferentially liberate a given fatty acid (e.g., C8:0 fatty acids) over all other fatty acids in the set of C8:0, C10:0, C12:0, C14:0, C16:0, C18:0, C18:1, and C18:2 fatty acids. The preference of the acyl-ACP preferring TE can be detected as a higher Vₘₐₓ (or a higher k_{cat}, or a higher V/K) in comparison to other non-preferred fatty acid-ACP substrates. The preference can be inferred from changes in fatty acid profile of a cell genetically engineered to overexpress the acyl-ACP preferring TE relative to a control cell that does not overexpress the acyl-ACP preferring TE.

Numbering of a given amino acid polymer or nucleic acid polymer “corresponds to” or is “relative to” the numbering of a selected amino acid polymer or nucleic acid polymer when the position of any given polymer component (e.g., amino acid, nucleotide, also referred to generically as a “residue”) is designated by reference to the same or to an equivalent position (e.g., based on an optimal alignment or a consensus sequence) in the selected amino acid or nucleic acid polymer, rather than by the actual numerical position of the component in the given polymer.

A “variant” is a polypeptide comprising a sequence which differs in one or more amino acid position(s) from that of a parent polypeptide sequence (e.g., by substitution, deletion, or insertion). A variant may comprise a sequence which differs from the parent polypeptides sequence in up to 40% of the total number of residues of the parent polypeptide sequence, such as in up to 40%, 35%, 30%, 25%, 20%, 15%, 10%, 9%, 8%, 7%, 6%, 5%, 4%, 3% 2% or 1% of the total number of residues of the parent polypeptide sequence. For example, a variant of a 400 amino acid polypeptide sequence comprises a sequence which differs in up to 40% of the total number of residues of the parent polypeptide sequence, that is, in up to 160 amino acid positions within the 400 amino acid polypeptide sequence (such as in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 78, 79, 80, 85, 90, 95, 100, 105, 110, 115, 120, 125, 130, 135, 140, 145, 150, 155, or 160 amino acid positions within the reference sequence.

“Naturally occurring” as applied to a composition that can be found in nature as distinct from being artificially produced by man. For example, a polypeptide or polynucleotide that is present in an organism (including viruses, bacteria, protozoa, insects, plants or mammalian tissue) that can be isolated from a source in nature and which has not been intentionally modified by man in the laboratory is naturally occurring. “Non-naturally occurring” (also termed “synthetic” or “artificial”) as applied to an object means that the object is not naturally-occurring—i.e., the object cannot be found in nature as distinct from being artificially produced by man.

A “cell oil” or “cell fat” shall mean a predominantly triglyceride oil obtained from an organism, where the oil has not undergone blending with another natural or synthetic oil, or fractionation so as to substantially alter the fatty acid profile of the oil. “Microbial oil” is to be understood herein as an oil obtained from microbial cells. Preferably, the microbial oil is an oil comprising triglycerides. In connection with an oil comprising triglycerides of a particular regiospecificity, the cell oil or cell fat has not been subjected to interesterification or other synthetic process to obtain that regiospecific triglyceride profile, rather the regiospecificity is produced naturally, by a cell or population of cells. For a cell oil or cell fat produced by a cell, the sterol profile of oil is generally determined by the sterols produced by the cell, not by artificial reconstitution of the oil by adding sterols in order to mimic the cell oil. In connection with a cell oil or cell fat, and as used generally throughout the present disclosure, the terms oil and fat are used interchangeably, except where otherwise noted. Thus, an “oil” or a “fat” can be liquid, solid, or partially solid at room temperature, depending on the makeup of the substance and other conditions. Here, the term “fractionation” means removing material from the oil in a way that changes its fatty acid profile relative to the profile produced by the organism, however accomplished. The terms “cell oil” and “cell fat” encompass such oils obtained from an organism, where the oil has undergone minimal processing, including refining, bleaching and/or degumming, which does not substantially change its triglyceride profile. A cell oil can also be a “noninteresterified cell oil”, which means that the cell oil has not undergone a process in which fatty acids have been redistributed in their acyl linkages to glycerol and remain essentially in the same configuration as when recovered from the organism.

The terms “lipid”, “neutral lipid”, “triglyceride”, “triacylglyceride”, “triacylglycerol”, “TAG”, and “triglyceride oil” are used interchangeably in the present disclosure, except where otherwise noted.

“Fatty acids” shall mean free fatty acids, fatty acid salts, or fatty acyl moieties in a glycerolipid. It will be understood that fatty acyl groups of glycerolipids can be described in terms of the carboxylic acid or anion of a carboxylic acid that is produced when the triglyceride is hydrolyzed or saponified.

As used herein, an oil is said to be “enriched” in one or more particular fatty acids if there is at least a 10% increase in the mass of that fatty acid in the oil relative to the non-enriched oil. For example, in the case of a cell expressing a heterologous or exogenous fatty acyl-ACP thioesterase gene described herein, the oil produced by the cell is said to be enriched in, e.g., C10 fatty acids, if the mass of these fatty acids in the oil is at least 10% greater than in oil produced by a cell of the same type that does not express the heterologous or exogenous fatty acyl-ACP thioesterase gene (e.g., wild type oil).

A “fatty acid profile” is the distribution of fatty acyl groups in the triglycerides of the oil without reference to attachment to a glycerol backbone. Fatty acid profiles are typically determined by conversion to a fatty acid methyl ester (FAME), followed by gas chromatography (GC) analysis with flame ionization detection (FID). The fatty acid profile can be expressed as one or more percent of a fatty acid in the total fatty acid signal determined from the area under the curve for that fatty acid. FAME-GC-FID measurement approximate weight percentages of the fatty acids.

A “triglyceride (TAG) profile” is the area percent distribution of each triglyceride in a triglyceride mixture generated by HPLC with RID (Refractive Index Detector) using AOCS method C3 5c-93, modified to include two columns as described in Example 1. As used herein, a percent of a specific triglyceride (e.g., tridecanoin) refers to the area percent of the triglyceride in a triglyceride mixture distribution, measured by the above method. The area percent of a triglyceride profile is approximately the mole percent distribution of each triglyceride in a triglyceride mixture. Typically, as is the case here, the possible regioisomers derived from having the same two or three different fatty acids at the different positions of the glycerol backbone are not resolved nor distinguished and are grouped and reported as one of the possible TAGs. Thus, in a triglyceride oil containing a mixture of CaOO (caprate-oleate-oleate), OCaO (oleate-caprate-oleate), and OOCa (oleate-oleate-caprate), the percent indicated for one of these TAGs would represent the sum of all possible isomers.

“Medium-long-chain triglyceride” or “MLCT” means a triglyceride, in which at least one medium chain fatty acid having 6 to 10 carbons (C6-C10) and at least one long chain fatty acid having 12 carbons or more are bound to the glycerol backbone.

“Microalgae” are microbial organisms that contain a chloroplast or plastid, and optionally that is capable of performing photosynthesis, or a prokaryotic microbial organism capable of performing photosynthesis. Microalgae include obligate photoautotrophs, which cannot metabolize a fixed carbon source as energy, as well as heterotrophs, which can live solely off of a fixed carbon source. Microalgae include unicellular organisms that separate from sister cells shortly after cell division, such as Chlamydomonas, as well as microbes such as, for example, Volvox, which is a simple multicellular photosynthetic microbe of two distinct cell types. Microalgae include eukaryotic Chlorophyceae such as Chlorella, Dunaliella, and Prototheca. Microalgae also include other microbial photosynthetic organisms that exhibit cell-cell adhesion, such as Agmenellum, Anabaena, and Pyrobotrys. Microalgae also include obligate heterotrophic microorganisms that have lost the ability to perform photosynthesis, such as certain dinoflagellate algae species and species of the genus Prototheca or Chlorella.

An “oleaginous” cell is a non-human cell capable of producing at least 20% lipid by dry cell weight, naturally or through recombinant or classical strain improvement. An “oleaginous microbe” or “oleaginous microorganism is a microbe, including a microalga that is oleaginous.

As used with respect to polypeptides or polynucleotides, the term “isolated” refers to a polypeptide or polynucleotide that has been separated from at least one other component that is typically present with the polypeptide or polynucleotide. Thus, a naturally occurring polypeptide is isolated if it has been purified away from at least one other component that occurs naturally with the polypeptide or polynucleotide. A recombinant polypeptide or polynucleotide is isolated if it has been purified away from at least one other component present when the polypeptide or polynucleotide is produced.

The terms “polypeptide” and “protein” are used interchangeably herein to refer a polymer of amino acids, and unless otherwise limited, include atypical amino acids that can function in a similar manner to naturally occurring amino acids.

The term “sequence”, as used in connection with a polypeptide or nucleic acid polymer refers to the order of monomers making up the polymer or the sub-polymer or fragment having that sequence.

A “subsequence” of an amino acid or nucleotide sequence is a portion of a larger sequence or the peptide or nucleic acid sub-polymer or fragment characterized by the portion of the larger sequence.

The term “percent sequence identity,” in the context of two or more amino acid or nucleic acid sequences, refers to two or more sequences or subsequences that are the same or have a specified percentage of amino acid residues or nucleotides that are the same, when compared and aligned for maximum correspondence, as measured using a sequence comparison algorithm or by visual inspection. For sequence comparison to determine percent nucleotide or amino acid identity, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence (e.g., SEQ ID NOs: 1-15), based on the designated program parameters. Optimal alignment of sequences for comparison can be conducted using the NCBI BLAST software (ncbi.nlm.nih.gov/BLAST/) set to default parameters. For example, to compare two nucleic acid sequences, one may use BLASTN program with its default parameters: (General Parameters: Max target sequences: 100; Expect threshold: 10; Word size: 28, Max matches in a query range: 0; Scoring parameters: Match/Mismatch Scores: 1, −2; Gap Costs: linear). For polypeptide sequence alignment and sequence identity calculations, BLASTP program can be used with its default parameters (General Parameters: Max target sequences: 100, Expect threshold: 10; Word size: 6; Max matches in a query range: 0; Scoring Parameters: Matrix=BLOSUM62; Gap costs: Existence=11, Extension=1; Compositional adjustments=Conditional compositional score). In certain embodiments, the sequence identity between two polypeptide sequences is determined using the Needleman-Wunsch algorithm (Needleman and Wunsch, 1970, J. Mol. Biol. 48: 443-453) as implemented in the Needle program of the EMBOSS package (https://www.ebi.ac.uk/Tools/psa/emboss_needle/) from the European Bioinformatics Institute, using its default parameters (Matrix: BLOSUM62; Gap Open: 10; Gap Extend: 0.5; End Gap Penalty: false; End Gap Open: 10; End Gap Extend: 0.5). In certain embodiments, the sequence identity between two nucleic acid sequences is determined using the Needleman-Wunsch algorithm described above using its default parameters (Matrix: DNAfull; Gap Open: 10; Gap Extend: 0.5; End Gap Penalty; false; End Gap Open: 10; End Gap Extend: 0.5). In certain embodiments, the sequence alignment of two or more sequences are performed using Clustal Omega or ClustalW using the suggested default parameters (Dealign input sequences: no; Mbed-like clustering guide-tree: yes; Mbed-like clustering iteration: yes; number of combined iterations: default(0); Max guide tree iterations: default; Max HMM iterations: default; Order: aligned).

As used with reference to polypeptides, the term “wild-type” refers to any polypeptide having an amino acid sequence present in a polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized.

The term “mutation” shall mean a change in a protein, polypeptide, or peptide sequence or subsequence produced by altering one or more nucleotides in a nucleotide coding for the protein, polypeptide, or peptide, however the alteration is obtained. For example, a mutation can be produced randomly, by PCR mutation, by synthesis of entire gene, or any other method.

The term “vector” is used herein to describe a DNA construct containing a polynucleotide. Such a vector can be propagated stably or transiently in a host cell. The vector can, for example, be a plasmid, a viral vector, or simply a potential genomic insert. Once introduced into a suitable host, the vector may replicate and function independently of the host genome, or may, in some instances, integrate into the host genome.

As used herein, the terms “expression vector” or “expression construct” or “expression cassette” refer to a nucleic acid construct, generated recombinantly or synthetically, with a series of specified nucleic acid elements that permit transcription of a particular nucleic acid in a host cell. The expression vector can be part of a plasmid, virus, or nucleic acid fragment. An “expression cassette” includes a coding nucleic acid (CDS) to be transcribed operably linked to a promoter and a 3′UTR. Optionally, and in the Examples below, the promoter of an expression cassette is a heterologous promoter.

“Exogenous gene” refers to a nucleic acid transformed into a cell. The exogenous gene may be from a different species (and so heterologous), or from the same species (and so homologous) relative to the cell being transformed. In the case of a homologous gene, it occupies a different location in the genome of the cell relative to the endogenous copy of the gene. The exogenous gene may be present in more than one copy in the cell. The exogenous gene may be maintained in a cell as an insertion into the genome or as an episomal molecule.

The term “heterologous” refers to amino acid subsequences that are not encoded by the naturally occurring gene. This can be accomplished in any way known in the art, including, e.g., swapping of individual domains with an altered and/or non-naturally occurring domain, introduction of point mutations, introduction of altered or non-naturally occurring subsequences, or deletion of single amino acid residues, subsequences and/or domains.

An “inducible promoter” is one that mediates transcription of an operably linked gene in response to a particular stimulus.

As used herein, the phrase “in operable linkage” refers to a functional linkage between two sequences, such a control sequence (typically a promoter) and the linked sequence. A promoter is in operable linkage with an exogenous gene if it can mediate transcription of the gene.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

An “allele” refers to a copy of a gene where an organism has multiple similar or identical gene copies, even if on the same chromosome. An allele may encode the same or similar protein.

As used herein, the term “recombinant” when used with reference, e.g., to a cell, or nucleic acid, protein, or vector, indicates that the cell, nucleic acid, protein or vector, has been modified by the introduction of an exogenous nucleic acid or protein or the alteration of a native nucleic acid or protein, or that the cell is derived from a cell so modified. Thus, recombinant cells express genes that are not found within the native (non-recombinant) form of the cell or express native genes that are otherwise abnormally expressed, over-expressed, under-expressed or not expressed at all. “Recombinant nucleic acid” as used herein refers to nucleic acid molecules that are initially synthesized through the use of laboratory methods, thereby creating nucleic acid sequences that are not normally found in nature. By using laboratory methods, recombinant nucleic acid molecules in operable linkage with different sequences (e.g., promoter, targeting sequence, etc.) is achieved. Thus an isolated nucleic acid, in a linear form, or an expression vector formed in vitro by ligating DNA molecules that are not normally joined, are both considered recombinant. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes herein. Similarly, a “recombinant protein” is a protein made using recombinant techniques, i.e., through the expression of a recombinant nucleic acid as depicted above.

A “transit peptide” is an amino acid sequence that directs the trafficking of a polypeptide fused to the signal sequence. In connection with plastidic cells expressing the polypeptide, the transit peptide may direct trafficking of the polypeptide to the plastid (i.e., a plastid targeting peptide).

The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands).

The term “host cell” refers to a cell capable of maintaining a vector either transiently or stably. Host cells include, without limitation, bacterial cells, yeast cells, insect cells, algal cells (e.g., microalgal cells), plant cells and mammalian cells. Other host cells known in the art, or which become known, are also suitable for use.

### DETAILED DESCRIPTION

### 1. Introduction

Provided are heterologous KAS enzymes that preferentially produce C10:0-ACP to facilitate the production of appreciable capric acid (C10:0) levels in host cells (e.g., microalgal cells such as Prototheca). The KAS enzyme variants can coordinate with a heterologous lipid biosynthesis enzyme, e.g., a thioesterase that preferentially hydrolyzes C10:0-acyl ACP substrates into capric acid and acyl carrier proteins (ACPs), so that the resulting capric acid can be incorporated into triglycerides (TAGs). We have previously identified both KAS (CpauKASIVa) gene from Cuphea paucipetala that is specific for C10:0 fatty acid production. See Intl. Appl. No. PCT/US15/39951, which is hereby incorporated herein by reference in its entirety. Here, we report on the identification of more active variants, CpauKASIVa^{T146S}, CpauKASIVa^{T146G}, and CpauKASIVa^{T146N} for strain engineering. By expressing one or more of these variants in combination with a thioesterase gene, the resulting transgenic strains can produce triglyceride oils with fatty acid profiles comprising a high level of C10:0 (e.g., almost 80% C10:0).

### 2. KASIVa Variants

The variant β-ketoacyl-ACP synthase (KAS) IVa enzymes (KASIVa) can be used in genetic constructs and genetically engineered oleaginous cells (e.g., plants, algae, microalgae) with one or more exogenous genes to produce fatty acids, acylglycerides, or derivatives thereof. For example, microalgae or oilseed crops that would naturally, or through genetic modification, produce high levels of triglycerides can be engineered (or further engineered) to express an exogenous variant KASIVa, which can catalyze the elongation of a medium-chain fatty acyl-ACP, e.g., from C8 to C10, preferentially produce C10:0-ACP, and/or facilitate the production of increased levels of C10 fatty acids, e.g., in comparison to a wild-type KASIVa. The fatty acids synthesized may be incorporated into acyl glycerides including triacylglycerides (TAGs, triglycerides). The triglycerides can be recovered or, through further enzymatic processing within the cell, or in vitro, yield other useful compounds.

Generally, the variant KASIVa enzymes described herein have preferential substrate specificity for medium-chain ACP-fatty acyl substrates (e.g., to promote the production of C8, C10, and/or C12, and/or C14 fatty acids, particularly C10 fatty acids). The variant KASIVa enzymes described herein catalyze the elongation of growing medium-chain fatty acyl-ACP, e.g., from 4 to 12 carbon atoms in length, particularly from 8 to 10 carbon atoms in length, and are categorized as EC 2.3.1.41 (β-ketoacyl-acyl-carrier-protein (ACP) synthase I).

In some embodiments, the non-natural or variant β-ketoacyl-ACP synthase (KAS) IVa enzymes (KASIVa) comprise at least about 80%, 85%, 90%, 91%, 92%, 93%, 94%, 95%, 96%, 97%, 98% or 99% identity to amino acid residues 34-523 of SEQ ID NO: 4 and comprises an X at the position corresponding to position 146; wherein X is an amino acid residue selected from the group consisting of glycine (G), asparagine (N), or serine (S), wherein the positions are with reference to SEQ ID NO: 4. In some embodiments, the X at position 146 is serine (S). In some embodiments, the X at position 146 is glycine (G). In some embodiments, the X at position 146 is asparagine (N).

In some embodiments, the non-natural or variant β-ketoacyl-ACP synthase (KAS) IVa enzyme (KASIVa) is encoded by a polynucleotide comprising at least about 80%, 85%, 90%, 91%, 92%, 93%, 94%, 95%, 96%, 97%, 98% or 99% identity to nucleic acid residues 100-1563 of SEQ ID NO: 7, SEQ ID NO: 9, or SEQ ID NO: 14. In some embodiments, the non-natural or variant β-ketoacyl-ACP synthase (KAS) IVa enzyme (KASIVa) is encoded by a polynucleotide comprising at least about 80%, 85%, 90%, 91%, 92%, 93%, 94%, 95%, 96%, 97%, 98% or 99% identity to SEQ ID NO: 7, SEQ ID NO: 9, or SEQ ID NO: 14.

In certain embodiments, provided is a fragment any of the above-described proteins or nucleic acids (including fragments of protein or nucleic acid variants), wherein the protein fragment has activity, e.g., to catalyze the elongation of a medium-chain fatty acyl-ACP, e.g., from C8 to C10, preferentially produce C10:0-ACP, and/or facilitate the production of increased levels of C10 fatty acids, e.g., in comparison to a wild-type KASIVa. Also contemplated are nucleic acid fragments encoding such protein fragments. In other embodiments, the fragment includes a domain of an acyl-ACP thioesterase that mediates a particular function, e.g., elongation of C8 to C10 fatty acids. Illustrative fragments can be produced by C-terminal and/or N-terminal truncations and include at least 50%, 55%, 60%, 65%, 70%, 75%, 80%, 85%, 90%, 95%, 96%, 97%, 98%, or 99% of the full-length sequences disclosed herein.

3. Co-Expression with C10-Preferring Thioesterases

In some embodiments, the variant KASIVa enzyme is co-expressed with a heterologous or exogenous fatty acyl-ACP thioesterase. In some embodiments, the thioesterase preferentially hydrolyzes C10-ACP substrates, e.g., catalyzes the production of increased levels of C10 fatty acids and/or has increased specificity for C10 fatty acids in comparison to a wild-type thioesterase. Illustrative C10-preferring thioesterases of use for co-expression include without limitation FATB from Lythraceae species, e.g., FATB from Cuphea species, including, e.g., Cuphea hookeriana FATB2 (ChFATB2), Cuphea paucipetala FATB1 (Cpau FATB1), Cuphea palustris FATB1 (Cpal FATB1, e.g., accession AAC49179), Cuphea ignea FATB1 (Cignea FATB1), Cuphea avigera FATB1 (Ca FATB1) (including K228M and K228I variants), Cuphea painteri FATB1 (Cpai FATB1), Cuphea procumbens FATB1 (CprocFATB1), Cuphea procumbens FATB3 (CprocFATB3), Cuphea crassiflora FATB1 (CcrasFATB1), Cuphea koehneana FATB3 (CkoeFATB3), Cuphea leptopoda FATB1 (CleptFATB1), Cuphea angustifolia FATB1 (CangFATB1), Cuphea llavea FATB1 (CllaFATB1), Cuphea lophostoma FATB1 (ClopFATB1), Cuphea PSR23 FatB3 (CuPSR23FATB3), Cuphea viscosissima FatB1 (CvisFATB1), and Cuphea glossostoma FatB1 (CgFATB1) and FATB thioesterases, variants, deletion mutants and chimeras described in, e.g., WO 2014/120829, WO 2014/151904, WO 2016/014968, and WO2016/044779, which are hereby incorporated herein by reference in their entireties for all purposes. A consensus C10:0 specific thioesterase sequence is provided in WO 2014/151904.

As disclosed in PCT/US2014/013676, we discovered that grafting the Cuphea avigera FATB1 (Ca FATB1) N-terminal specificity domain onto the Cuphea hookeriana FATB2 improves activity and C8-C10 ratio. Prototheca moriformis transformants expressing Ch FATB2 H163Y, L186P (D3130) mutants exhibited about 2 fold increase in the average C8-C10 sum as well as a shift in fatty acid profile specificity relative to the wild-type Ch FATB2 (D3042).

In some embodiments, the encoded thioesterase comprises at least about 80%, 85%, 90%, 91%, 92%, 93%, 94%, 95%, 96%, 97%, 98% or 99% identity to amino acid residues 39-392 of SEQ ID NO: 5.

4. Co-Expression with Other Lipid Biosynthesis Enzymes

In some embodiments, the variant KASIVa enzyme is co-expressed with one or more heterologous or exogenous lipid biosynthesis enzymes. In some embodiments, the variant KASII enzyme is co-expressed with one or more heterologous or exogenous lipid biosynthesis enzymes selected from the group consisting of a fatty acyl thioesterase A (FATA), a fatty acyl thioesterase B (FATB), a 1-acylglycerol-3-phosphate O-acyltransferase (LPAAT), a glycerol-3-phosphate acyltransferase (GPAT), an acyl CoA:diacylglycerol acyltransferase (DGAT), and a fatty acid elongase (FAE), a long-chain acyl-CoA synthetase (LACS). In some embodiments, the host cell further comprises one or more exogenous or heterologous enzymes, such as a sucrose invertase and a 4-amino-5-hydroxymethyl-2-methylpyrimidine phosphate synthase (THIC). Recombinant expression of heterologous or exogenous lipid biosynthesis enzymes is described, e.g., in U.S. Patent Publ. No. 2014/0178950, which is incorporated herein by reference in its entirety for all purposes. For example, one or more polynucleotides encoding one or more of the aforementioned lipid biosynthesis enzymes can be used in a variety of genetic constructs including plasmids or other vectors for expression or recombination in a host cell. The genes can be codon optimized for expression in a target host cell. The genes can be included in an expression cassette that includes a promoter (e.g., a heterologous promoter) and downstream regulatory element. The vector can include flanking sequences for homologous recombination. For example, the vector can cause insertion into a chromosome of the host cell, where it can be stably expressed. The proteins produced by the genes can be used in vivo or in purified form. In an embodiment, an expression cassette comprises a homologous promoter, a CDS operable to express one or more lipid biosynthesis enzymes and a 3′UTR. The 3′UTR can comprise a polyadenylation site.

In some embodiments, one or more lipid biosynthesis enzymes endogenous to the host cell selected from the group consisting of a fatty acyl thioesterase A (FATA), a fatty acyl thioesterase B (FATB), a 1-acylglycerol-3-phosphate O-acyltransferase (LPAAT), a glycerol-3-phosphate acyltransferase (GPAT), an acyl CoA:diacylglycerol acyltransferase (DGAT), a fatty acid elongase (FAE) and a long-chain acyl-CoA synthetase (LACS) are deleted, knocked out or knocked down. For example, one or more polynucleotides encoding one or more of the aforementioned lipid biosynthesis enzymes can also be used to prepare antisense, or inhibitory RNA (e.g., RNAi or hairpin RNA) to inhibit complementary genes in the microalgal host cell. For example, armed with the knowledge of a gene sequence encoding one of the aforementioned proteins, one can engineer a microalgal host cells with the same or similar gene to express an RNAi construct, gene knockout, knockdown, point mutation, or the like, and thereby reduce the expression and/or activity of one or more of the enzymes in the microalgal host cell. As a result, the microalgae can produce an oil with an altered fatty acid profile in which the mean chain length is decreased or increased, depending on the presence of other fatty acid synthesis genes. In some embodiments, a mutation (including knockout) or inhibition (e.g., using antisense or RNAi) of one or more endogenous desaturase genes (e.g., a stearoyl-ACP desaturase or fatty acid desaturase including a delta 12 fatty acid desaturase) can reduce or eliminate desaturase activity to produce a more fully saturated triglyceride profile.

Depending on the desired properties of the triglyceride molecule to be produced, one or more genes encoding enzymes that utilize fatty acids or fatty acyl molecules as substrates to produce triglyceride molecules may be attenuated or over-expressed in the host cell (e.g., microalga), for example using RNAi, hairpin constructs, knockdowns, double or single knockouts or replacement (e.g., replacing an endogenous gene with a heterologous gene).

### 5. Codon-Bias for Improved Expression in Microalgal Host Cells

DNA encoding a polypeptide to be expressed in a microorganism, e.g., a KASIVa variant, optionally with an exogenous lipid biosynthesis enzyme, e.g., a fatty acyl-ACP thioesterase, and selectable marker can be codon-optimized cDNA. Methods of recoding genes for expression in microalgae are described in U.S. Pat. No. 7,135,290. Additional information for codon optimization is available, e.g., at the Codon Usage Database at kazusa.or.jp/codon/. The table for Prototheca preferred codon usage is also provided in U.S. Patent Publ. No. 2012/0283460. Preferred codon usage in Prototheca and Chlorella protothecoides is provided in Tables A and B.

TABLE A
Preferred codon usage in Prototheca strains.

| Amino | | Usage | Amino | | Usage |
|---|---|---|---|---|---|
| Acid | Codon | Frequency | Acid | Codon | Frequency |
| Ala | GCG | 36% | Leu | TTG | 4% |
| | GCA | 7% | | TTA | 0% |
| | GCT | 11% | | CTG | 61% |
| | GCC | 46% | | CTA | 3% |
| Arg | AGG | 6% | | CTT | 6% |
| | AGA | 2% | | CTC | 26% |
| | CGG | 18% | Met | ATG | 100% |
| | CGA | 8% | Phe | TTT | 29% |
| | CGT | 9% | | TTC | 71% |
| | CGC | 57% | Pro | CCG | 29% |
| Asn | AAT | 4% | | CCA | 9% |
| | AAC | 96% | | CCT | 13% |
| Asp | GAT | 12% | | CCC | 49% |
| | GAC | 88% | Ser | AGT | 3% |
| Cys | TGT | 10% | | AGC | 22% |
| | TGC | 90% | | TCG | 28% |
| Gln | CAG | 82% | | TCA | 6% |
| | CAA | 18% | | TCT | 10% |
| Glu | GAG | 96% | | TCC | 31% |
| | GAA | 4% | Thr | ACG | 38% |
| Gly | GGG | 12% | | ACA | 5% |
| | GGA | 7% | | ACT | 5% |
| | GGT | 10% | | ACC | 52% |
| | GGC | 71% | Ty | TAT | 5% |
| His | CAT | 21% | | TAC | 95% |
| | CAC | 79% | Trp | TGG | 100% |
| Ile | ATA | 1% | Val | GTG | 50% |
| | ATT | 8% | | GTA | 1% |
| | ATC | 91% | | GTT | 6% |
| Lys | AAG | 98% | | GTC | 43% |

| | | | |
|---|---|---|---|
| AAA | 2% | Stop | TGA/TAG/TAA |

TABLE B
Preferred codon usage in Chlorella protothecoides.

| Amino | Preferred | Amino | Preferred |
|---|---|---|---|
| Acid | Codon | Acid | Codon |
| Arg | CGC | Lys | AAG |
| Ala | GCC | Met | ATG |
| Asn | AAC | Phe | TTC |
| Asp | GAC | Pro | CCC |
| Cys | TGC | Ser | TCC |
| Gln | CAG | Thr | ACC |
| Glu | GAG | Trp | TGG |
| Gly | GGC | Tyr | TAC |
| His | CAC | Val | GTG |
| Ile | ATC | Stop | TGA |
| Leu | CTG | | |

In various embodiments, the nucleic acids encoding the KASIVa variants, and optionally the co-expressed exogenous lipid biosynthesis enzyme, e.g., fatty acyl-ACP thioesterase, can be codon biased for improved expression in a target host cell. For expression in a Prototheca or a Chlorella host cell, the encoding polynucleotide can be recoded, using the preferred codons identified in Tables A or B, respectively. For example, in some embodiments, at least 60%, 65%, 70%, 75%, 80%, 85%, 90%, 95%, or 100% of the codons used in the encoding polynucleotide can be the most preferred codon according to Tables A or B. In some embodiments, at least 60%, 65%, 70%, 75%, 80%, 85%, 90%, 95%, or 100% of the codons used in the encoding polynucleotide can be the first or second most preferred codon according to Tables A and B. In some embodiments, the non-natural or variant 3-ketoacyl-ACP synthase (KAS) IVa enzyme (KASIVa) is encoded by a polynucleotide comprising at least about 80%, 85%, 90%, 91%, 92%, 93%, 94%, 95%, 96%, 97%, 98% or 99% identity to nucleic acid residues 100-1563 of SEQ ID NO: 7, SEQ ID NO: 9, or SEQ ID NO: 14. In some embodiments, the non-natural or variant β-ketoacyl-ACP synthase (KAS) IVa enzyme (KASIVa) is encoded by a polynucleotide comprising at least about 80%, 85%, 90%, 91%, 92%, 93%, 94%, 95%, 96%, 97%, 98% or 99% identity to SEQ ID NO: 7, SEQ ID NO: 9, or SEQ ID NO: 14.

### 6. Expression and Targeting to Plastids

Heterologous or exogenous proteins expressed in the nuclear genome of Prototheca can be targeted to the plastid using plastid targeting signals. Plastid targeting sequences endogenous to Chlorella are known, such as genes in the Chlorella nuclear genome that encode proteins that are targeted to the plastid; see for example GenBank Accession numbers AY646197 and AF499684, and in one embodiment, such control sequences are used in the vectors described herein, e.g., to target expression of a protein to a Prototheca plastid.

The Examples below describe the use of algal plastid targeting sequences to target heterologous proteins to the correct compartment in the host cell. cDNA libraries were made using Prototheca moriformis and Chlorella protothecoides cells and are described in the Examples of U.S. Patent Publ. No. 2012/0283460 and in PCT Application No. PCT/US2009/066142. Amino acid sequences of the algal plastid targeting sequences identified from the cDNA libraries useful plastid targeting of recombinantly expressed variant KASIV enzymes are provided in U.S. Patent Publ. No. 2012/0283460 and herein. In some embodiments, the plastid transit peptide comprises an amino acid sequence selected from the group consisting of MATASTFSAFNARCGDLRRSAGSGPRRPARPLPVRGRA (SEQ ID NO: 10), SGPRRPARPLPVR (SEQ ID NO: 16), SGPRRPARPLPVRAAIASEVPVATTSPR (SEQ ID NO: 17), RPARPLPVRGRA (SEQ ID NO: 18), RPARPLPVRAAIASEVPVATTSPR (SEQ ID NO: 19), RCGDLRRSAGSGPRRPARPLPVRGRA (SEQ ID NO: 20), RCGDLRRSAGSGPRRPARPLPVRAAIASEVPVATTSPR (SEQ ID NO: 21), PARPLPVR (SEQ ID NO: 22), PARPLPVRAAIASEVPVATTSPR (SEQ ID NO: 23), RRPARPLPVR (SEQ ID NO: 24), and RRPARPLPVRAAIASEVPVATTSPR (SEQ ID NO: 25). In some embodiments, the plastid transit peptide comprises at least about 80%, 85%, 90%, 91%, 92%, 93%, 94%, 95%, 96%, 97%, 98% or 99% identity to amino acid residues 1-33 of SEQ ID NO: 3, amino acid residues 1-33 of SEQ ID NO: 4, SEQ ID NO: 10 or SEQ ID NO: 11. In some embodiments, the plastid transit peptide is encoded by a polynucleotide comprising at least about 80%, 85%, 90%, 91%, 92%, 93%, 94%, 95%, 96%, 97%, 98% or 99% identity to SEQ ID NO: 12.

Where novel KASIV variants are disclosed here, it will be understood that a variety of heterologous plastid transit peptides can be used. In other words, the non-targeting peptide domain is more highly conserved. Accordingly, embodiments described herein feature the novel KASIV enzymatic domain with or without a plastid targeting sequence. For example, where a percent identity to a novel KASIV gene is given herein, the same identity can be applied (where specified) to the same sequence absent the targeting peptide. A substitute targeting peptide can optionally be used in connection with such a sequence.

### 7. Host Cells

Any species of organism that produces suitable lipids or triglycerides can be used, although microorganisms that naturally produce high levels of suitable triglycerides are preferred. Considerations for the selection of microorganisms include, in addition to production of suitable lipids or triglycerides for production of oils and oleochemicals: (1) high lipid content as a percentage of dry cell weight; (2) ease of growth; (3) ease of genetic engineering; and (4) ease of biomass processing. In particular embodiments, the wild-type, classically improved or genetically engineered microorganism yields cells that are at least 40%, at least 45%, at least 50%, at least 55%, at least 60%, at least 65%, or at least 70% or more lipid as a percentage of their dry cell weight. Preferred organisms grow heterotrophically (on sugars in the absence of light) or can be engineered to do so using, for example, methods disclosed herein. The ease of transformation and availability of selectable markers and promoters, constitutive or inducible, that are functional in the microorganism affect the ease of genetic engineering. Examples of selectable markers useful in microalgae include sucrose invertase, alpha galactosidase (for selection on melibiose) and antibiotic resistance genes. Processing considerations can include, for example, the availability of effective means for lysing the cells.

### Microalgae

In some embodiments, the microorganism is a microalga. Non-limiting examples of microalgae that can be used for expression of variant KASIV enzymes include, e.g., Achnanthes orientalis, Agmenellum, Amphiprora hyaline, Amphora coffeiformis, Amphora coffeiformis linea, Amphora coffeiformis punctata, Amphora coffeiformis taylori, Amphora coffeiformis tenuis, Amphora delicatissima, Amphora delicatissima capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri subsalsum, Chaetoceros sp., Chlorella anitrata, Chlorella Antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora (strain SAG 37.88), Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides (including any of UTEX strains 1806, 411, 264, 256, 255, 250, 249, 31, 29, 25), Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris f. tertia, Chlorella vulgaris var. autotrophica, Chlorella vulgaris var. viridis, Chlorella vulgaris var. vulgaris, Chlorella vulgaris var. vulgaris f. tertia, Chlorella vulgaris var. vulgaris f. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena, Franceia sp., Fragilaria crotonensis, Fragilaria sp., Gleocapsa sp., Gloeothamnion sp., Hymenomonas sp., Isochrysis aff galbana, Isochrysis galbana, Lepocinclis, Micractinium, Micractinium (UTEX LB 2614), Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, ParaChlorella kessleri, Pascheria acidophila, Pavlova sp., Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, PseudoChlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Tetraedron, Tetraselmis sp., Tetraselmis suecica, Thalassiosira weissflogii, and Viridiella fridericiana.

Illustrative host cells feature oleaginous cells that yield altered fatty acid profiles and/or altered regiospecific distribution of fatty acids in glycerolipids and products produced by the cells. Examples of oleaginous cells include microbial cells having a type II lipid biosynthesis pathway, including plastidic oleaginous cells such as those of oleaginous algae. Specific examples of cells include heterotrophic or obligate heterotophic microalgae of the phylum Chlorpophya, the class Trebouxiophytae, the order Chlorellales, or the family Chlorellacae. Examples of oleaginous microalgae are provided in Published PCT Patent Applications WO2008/151149, WO2010/06032, WO2011/150410, and WO2011/150411, including species of Chlorella and Prototheca, a genus comprising obligate heterotrophs. The oleaginous cells can be, for example, capable of producing 25, 30, 40, 50, 60, 70, 80, 85, or about 90% lipid by cell weight, ±5%. The above mentioned publications also disclose methods for cultivating such cells and extracting oil, especially from microalgal cells; such methods are applicable to the cells disclosed herein. In any of the embodiments described herein, the cells can be heterotrophic cells comprising an exogenous sucrose invertase gene so as to allow the cells to produce oil from a sucrose feedstock.

Illustrative embodiments of host cells include recombinant oleaginous cells expressing one or more exogenous genes encoding fatty acid or triacylglyceride biosynthesis enzymes. As a result, some embodiments feature natural oils never before obtainable in a natural oil. In some cases, the natural oils were not obtainable from a non-plant or non-seed oil, or not obtainable at all.

The oleaginous cells produce a storage oil, which may be stored in storage vesicles of the cell. A raw oil may be obtained from the cells by disrupting the cells and isolating the oil. The oils produced maybe refined, bleached and deodorized (RBD) as known in the art or as described in WO2010/120939. The raw or RBD oils may be used in a variety of food, chemical, and industrial products or processes. After recovery of the oil, a valuable residual biomass remains. Uses for the residual biomass include the production of paper, plastics, absorbents, adsorbents, as animal feed, for human nutrition, or for fertilizer.

Where a fatty acid profile of a triglyceride cell oil is given, it will be understood that this refers to a nonfractionated sample of the storage oil extracted from the cell analyzed under conditions in which phospholipids have been removed or with an analysis method that is substantially insensitive to the fatty acids of the phospholipids (e.g. using chromatography and mass spectrometry). Because the cells are oleaginous, in some cases the storage oil will constitute the bulk of all the triglycerides in the cell.

In some embodiments, the host cell is a plastidic cell, e.g., a heterotrophic microalga of the phylum Chlorpophya, the class Trebouxiophytae, the order Chlorellales, or the family Chlorellacae. In some embodiments, the cell is oleaginous and capable of accumulating at least 40% lipid by dry cell weight. The cell can be an obligate heterotroph, such as a species of Prototheca, including Prototheca moriformis or Prototheca zopfii. The nucleic acid encoding the variant KASIV enzymes described herein can also be expressed in autotrophic algae or plants. Optionally, the cell is capable of using sucrose to produce oil and a recombinant invertase gene may be introduced to allow metabolism of sucrose, as described in PCT Publications WO2008/151149, WO2010/06032, WO2011/150410, WO2011/150411, and international patent application PCT/US12/23696. The invertase may be codon-biased and integrated into a chromosome of the cell, as may all of the genes mentioned here. Codon usage for different algal and plant species of interest is known in the art and can be found, e.g., on the internet at the Codon Usage Database at kazusa.or.jp/codon/.

The polynucleotides encoding the variant KASIV described herein further can be expressed in a wide variety of plant and microalgal host cells. Of particular interest are plant cells of plants involved in the production of vegetable oils for edible and industrial uses, including e.g., temperate oilseed crops. Plants of interest include, but are not limited to, rapeseed (Canola and High Erucic Acid varieties), sunflower, safflower, cotton, Cuphea, soybean, peanut, coconut and oil palms, and corn. See, U.S. Pat. Nos. 5,850,022; 5,723,761; 5,639,790; 5,807,893; 5,455,167; 5,654,495; 5,512,482; 5,298,421; 5,667,997; and U.S. Pat. Nos. 5,344,771; 5,304,481.

### 8. Methods of Culturing Microorganisms

Microorganisms are cultured both for purposes of conducting genetic manipulations and for subsequent production of oil or triglycerides (TGs, triacylglycerols, TAGs, or triacylglycerides). The former type of culture is conducted on a small scale and initially, at least, under conditions in which the starting microorganism can grow. For example, if the starting microorganism is a photoautotroph the initial culture is conducted in the presence of light. The culture conditions can be changed if the microorganism is evolved or engineered to grow independently of light. Culture for purposes of oil or triglyceride production is usually conducted on a large scale. Preferably a fixed carbon source is present. The culture can also be exposed to light some or all of the time.

Microalgae can be cultured in liquid media. The culture can be contained within a bioreactor. Optionally, the bioreactor does not allow light to enter. Alternatively, microalgae can also be cultured in photobioreactors that contain a fixed carbon source and allow light to strike the cells. Exposure of microalgae cells to light, even in the presence of a fixed carbon source that the cells transport and utilize (i.e., mixotrophic growth), nonetheless accelerates growth compared to culturing cells in the dark. Culture condition parameters can be manipulated to increase or improve total triglyceride production, the combination of triglyceride species produced, and/or production of a triglyceride species. In some instances it is preferable to culture cells in the dark, such as, for example, when using extremely large (e.g., 10,000 L, 40,000 L, 100,000 L, 500,000 L, or larger, bioreactors) fermentors that do not allow light to strike the culture.

Microalgal culture media typically contain components such as a fixed nitrogen source, trace elements, vitamins (e.g., thiamine), optionally a buffer for pH maintenance, and phosphate. Other components can include a fixed carbon source such as acetate or glucose, and salts such as sodium chloride, particularly for seawater microalgae. Examples of trace elements include zinc, boron, cobalt, copper, manganese, and molybdenum in, for example, the respective forms of ZnCl₂, H₃BO₃, CoCl₂·6H₂O, CuCl₂·2H₂O, MnCl₂·4H₂O and (NH₄)₆Mo₇O₂₄·4H₂O.

For organisms able to grow on a fixed carbon source, the fixed carbon source can be, for example, glucose, fructose, sucrose, galactose, xylose, mannose, rhamnose, N-acetylglucosamine, glycerol, floridoside, and/or glucuronic acid. The one or more carbon source(s) can be supplied at a concentration of at least about 50 μM, at least about 100 μM, at least about 500 μM, at least about 5 mM, at least about 50 mM, and at least about 500 mM, of one or more exogenously provided fixed carbon source(s). Some microalgae species can grow by utilizing a fixed carbon source such as glucose or acetate in the absence of light. Such growth is known as heterotrophic growth. For Chlorella and/or Prototheca, for example, heterotrophic growth results in high production of biomass and accumulation of high lipid content in cells.

Some microorganisms naturally grow on or can be engineered to grow on a fixed carbon source that is a heterogeneous source of compounds such as municipal waste, secondarily treated sewage, wastewater, and other sources of fixed carbon and other nutrients such as sulfates, phosphates, and nitrates. The sewage component serves as a nutrient source in the production of triglycerides, and the culture provides an inexpensive source of triglycerides.

Other culture parameters can also be manipulated, such as the pH of the culture media, the identity and concentration of trace elements and other media constituents.

### Heterotrophic Growth

As an alternative to photosynthetic growth of microorganisms, some microorganisms can be cultured under heterotrophic growth conditions in which a fixed carbon source provides energy for growth and lipid accumulation.

Provided are significantly improved culture parameters incorporating the use of glycerol for fermentation of multiple genera of both eukaryotic and prokaryotic microbes, including microbes of the genera Prototheca, Chlorella, Navicula, Scenedesmus, and Spirulina. Standard methods for the growth and propagation of Chlorella and/or Prototheca are known (see for example Miao and Wu, J. Biotechnology, 2004, 11:85-93 and Miao and Wu, Biosource Technology (2006) 97:841-846). In addition, multiple species of Chlorella and/or Prototheca and multiple strains within a species can be grown, e.g., in the presence of a sugar (e.g., glucose, sucrose, xylose) and/or glycerol, including glycerol byproduct from biodiesel transesterification.

For oil production, cells, including recombinant cells described herein, are preferably cultured or fermented in large quantities. The culturing may be in large liquid volumes, such as in suspension cultures as an example. Other examples include starting with a small culture of cells which expand into a large biomass in combination with cell growth and propagation as well as oil production. Bioreactors or steel fermentors can be used to accommodate large culture volumes. A fermentor similar to those used in the production of beer and/or wine is suitable, as are extremely large fermentors used in the production of ethanol.

Appropriate nutrient sources for culture in a fermentor are provided. These include raw materials such as one or more of the following: a fixed carbon source such as glucose, corn starch, depolymerized cellulosic material, sucrose, sugar cane, sugar beet, lactose, milk whey, or molasses; a fat source, such as fats or vegetable oils; a nitrogen source, such as protein, soybean meal, cornsteep liquor, ammonia (pure or in salt form), nitrate or nitrate salt, or molecular nitrogen; and a phosphorus source, such as phosphate salts. Additionally, a fermentor allows for the control of culture conditions such as temperature, pH, oxygen tension, and carbon dioxide levels. Optionally, gaseous components, like oxygen or nitrogen, can be bubbled through a liquid culture. Other starch (polymerized glucose) sources such as wheat, potato, rice, and sorghum. Other carbon sources include process streams such as technical grade glycerol, black liquor, organic acids such as acetate, and molasses. Carbon sources can also be provided as a mixture, such as a mixture of sucrose and depolymerized sugar beet pulp.

A fermentor can be used to allow cells to undergo the various phases of their growth cycle. As an example, an inoculum of oil-producing cells can be introduced into a medium followed by a lag period (lag phase) before the cells begin growth. Following the lag period, the growth rate increases steadily and enters the log, or exponential, phase. The exponential phase is in turn followed by a slowing of growth due to decreases in nutrients and/or increases in toxic substances. After this slowing, growth stops, and the cells enter a stationary phase or steady state, depending on the particular environment provided to the cells.

Oil production by cells disclosed herein can occur during the log phase or thereafter, including the stationary phase wherein nutrients are supplied, or still available, to allow the continuation of oil production in the absence of cell division.

In some embodiments, microorganisms grown using conditions described herein and comprise at least about 20% by weight of lipid, preferably at least about 40% by weight, at least about 50% by weight, and more preferably at least about 60% by weight, even more preferably at least about 70%, 75%, 80% or 85% by weight.

In one heterotrophic growth method, sucrose, produced by example from sugar cane or sugar beet, is used as a feedstock. Oil production can be facilitated or made more efficient through the engineering of microbes such as Chlorella and/or Prototheca, to utilize sucrose as a carbon source. For example, expression of a sucrose transporter and a sucrose invertase allows Chlorella and/or Prototheca to transport sucrose into the cell from the culture media and hydrolyze sucrose to yield glucose and fructose. Optionally, a fructokinase can be expressed as well in instances where endogenous hexokinase activity is insufficient for maximum phosphorylation of fructose. Examples of suitable sucrose transporters are Genbank accession numbers CAD91334, CAB92307, and CAA53390. Examples of suitable sucrose invertases are Genbank accession numbers CAB95010, NP012104 and CAA06839. Examples of suitable fructokinases are Genbank accession numbers P26984, P26420 and CAA43322. Vectors for transformation of microalgae, including Chlorella and/or Prototheca, encoding one or more of such genes can be designed as described herein.

Secretion of a sucrose invertase can obviate the need for expression of a transporter that can transport sucrose into the cell. This is because a secreted invertase catalyzes the conversion of a molecule of sucrose into a molecule of glucose and a molecule of fructose, both of which can be transported and utilized by microbes disclosed herein. For example, expression of a sucrose invertase with a secretion signal generates invertase activity outside the cell. See Hawkins et al., Current Microbiology Vol. 38 (1999), pp. 335-341 for examples of secretion signals active in Chlorella and/or Prototheca. Expression of such a protein, as enabled by the genetic engineering methodology disclosed herein, allows cells already capable of utilizing extracellular glucose as an energy source to utilize sucrose as an extracellular energy source. Chlorella and/or Prototheca cells can use both extracellular fructose and extracellular glucose as an energy source, secretion of an invertase can provide the sole catalytic activity necessary for use of sucrose as an efficient, inexpensive energy source.

For example, Chlorella and/or Prototheca cells can be engineered with a sucrose invertase gene under the regulatory control of one of three promoters (Cauliflower mosaic virus 35S promoter (CMV), Chlorella virus promoter (CV), or Chlorella HUP1 promoter (HUP 1)). The sucrose invertase gene used in this example comprises codon-bias to the S. cerevisiae SUC2 gene to improve expression in a C. protothecoides host cell. Expression of a secretable sucrose invertase, such as that described herein, permits the use of molasses, sugar cane juice, and other sucrose-containing feedstocks for cell fermentation.

Alternatively, a sucrose invertase can also be expressed intracellularly in cells that express a sucrose transporter, as well as in cells that express any carbohydrate transporter that allows sucrose to enter the cell.

Bioreactors can be employed for use in heterotrophic growth methods. As will be appreciated, provisions made to make light available to the cells in photosynthetic growth methods are unnecessary when using a fixed-carbon source in the heterotrophic growth methods described herein.

The specific examples of process conditions and heterotrophic growth methods described herein can be combined in any suitable manner to improve efficiencies of microbial growth and/or lipid production. Additionally, conditions and heterotrophic growth methods are useful in the selection and/or genetic engineering of microbes, such as microalgae, to produce microbes that are even more suitable for use in the above-described methods. For example, the microbes having a greater ability to utilize any of the above-described feedstocks for increased proliferation and/or lipid (e.g., fatty acid) production are within the scope of the compositions and methods described herein.

### Growth Media

Microorganisms useful in accordance with the methods described herein are found in various locations and environments throughout the world. As a consequence of their isolation from other species and their resulting evolutionary divergence, the particular growth medium for optimal growth and generation of triglyceride constituents can be difficult to predict. In some cases, certain strains of microorganisms may be unable to grow on a particular growth medium because of the presence of some inhibitory component or the absence of some essential nutritional requirement required by the particular strain of microorganism.

Solid and liquid growth media are generally available from a wide variety of sources, and instructions for the preparation of particular media that is suitable for a wide variety of strains of microorganisms can be found, for example, online at utex.org/, a site maintained by the University of Texas at Austin for its culture collection of algae (UTEX). For example, various fresh water and salt water media are provided in U.S. Patent Publ. No. 2012/0288930, hereby incorporated herein by reference in its entirety for all purposes.

In a particular example, a medium suitable for culturing Chlorella and/or Prototheca cells comprises Proteose Medium. This medium is suitable for axenic cultures, and a 1 L volume of the medium (pH .about.6.8) can be prepared by addition of 1 g of proteose peptone to 1 liter of Bristol Medium. Bristol medium comprises 2.94 mM NaNO₃, 0.17 mM CaCl₂2H₂O, 0.3 mM MgSO₄7H₂O, 0.43 mM, 1.29 mM KH₂PO₄, and 1.43 mM NaCl in an aqueous solution. For 1.5% agar medium, 15 g of agar can be added to 1 L of the solution. The solution is covered and autoclaved, and then stored at a refrigerated temperature prior to use.

Other suitable media for use with the methods described herein can be readily identified by consulting the URL identified above, or by consulting other organizations that maintain cultures of microorganisms, such as SAG, CCAP, or CCALA. SAG refers to the Culture Collection of Algae at the University of Gottingen (Gottingen, Germany), CCAP refers to the culture collection of algae and protozoa managed by the Scottish Association for Marine Science (Scotland, United Kingdom), and CCALA refers to the culture collection of algal laboratory at the Institute of Botany (ccala.butbn.cas.cz/, Czech Republic).

### Increasing Production of Lipids

Process conditions can be adjusted to increase the production of lipids suitable for a particular use and/or to reduce production cost. For example, in certain embodiments, an oleaginous cell (e.g., a plant, an algae, a microalga) is cultured in the presence of a limiting concentration of one or more nutrients, such as, for example, carbon and/or nitrogen, phosphorous, or sulfur, while providing an excess of fixed carbon energy such as glucose. Nitrogen limitation tends to increase microbial lipid production over microbial lipid production in a culture in which nitrogen is provided in excess. In particular embodiments, the increase in lipid production is at least about: 10%, 20%, 30%, 40%, 50%, 75%, 100%, 200%, 300%, 400%, or 500%. The oleaginous cells (e.g., plant cells, algae cells, microalgal cells) can be cultured in the presence of a limiting amount of a nutrient for a portion of the total culture period or for the entire period. In particular embodiments, the nutrient concentration is cycled between a limiting concentration and a non-limiting concentration at least twice during the total culture period.

In another embodiment, lipid production is increased by culturing oleaginous cells or an oleaginous organism (e.g., plants, algae, microalgae) in the presence of one or more cofactor(s) for a lipid pathway enzyme (e.g., a fatty acid synthetic enzyme). Generally, the concentration of the cofactor(s) is sufficient to increase microbial lipid (e.g., fatty acid) production over microbial lipid production in the absence of the cofactor(s). In a particular embodiment, the cofactor(s) are provided to the culture by including in the culture oleaginous cells (e.g., plant cells, algae cells, microalgae cells) containing an exogenous gene encoding the cofactor(s). Alternatively, cofactor(s) may be provided to a culture by including an oleaginous cell (e.g., a plant, an algae, a microalgae) containing an exogenous gene that encodes a protein that participates in the synthesis of the cofactor. In certain embodiments, suitable cofactors include any vitamin required by a lipid pathway enzyme, such as, for example: biotin, pantothenate. Genes encoding cofactors suitable for use in the present compositions and methods or that participate in the synthesis of such cofactors are well known and can be introduced into oleaginous cells (e.g., plant cells, algae cells, microalgal cells), using constructs and techniques such as those described above and herein.

In some embodiments, the cells can be fully auxotrophic or partially auxotrophic (i.e., synthetic sickness or lethality) with respect to one or more types of fatty acid. The cells are cultured with supplementation of the fatty acid(s) so as to increase the cell number, then allowing the cells to accumulate oil (e.g., to at least 40% by dry cell weight). Alternatively, the cells comprise a regulatable fatty acid synthesis gene that can be switched in activity based on environmental conditions and the environmental conditions during a first, cell division, phase favor production of the fatty acid and the environmental conditions during a second, oil accumulation, phase disfavor production of the fatty acid.

As a result of applying either of these supplementation or regulation methods, a cell oil may be obtained from the cell that has low amounts of one or more fatty acids essential for optimal cell propagation. Specific examples of oils that can be obtained include those low in stearic, linoleic and/or linolenic acids. Optionally, the cells are oleaginous plastidic microbes such as those of the division Chlorophyta.

Accordingly, in some embodiments, provided are methods for producing an oil or fat. The method comprises cultivating a recombinant oleaginous cell in a growth phase under a first set of conditions that is permissive to cell division so as to increase the number of cells due to the presence of a fatty acid, cultivating the cell in an oil production phase under a second set of conditions that is restrictive to cell division but permissive to production of an oil that is enriched in C8 and/or C10 fatty acids. The cell can be cultivated heterotrophically. In some embodiments, the cell can be a microalgal cell and may produce at least 40%, 50%, 60%, 70%, 80%, or 90% lipid by dry cell weight.

9. Oils with Non-Naturally Occurring Fatty Acid Profiles

Oils disclosed herein can be prepared from the microbial cell biomass by using extraction methods well-known in the art. The microbial biomass may be concentrated from the fermentation broth, and optionally dried prior to cell lysis. Alternatively, cells can be lysed without separation from some or all of the fermentation broth when the fermentation is complete. Any suitable extraction methods can be used. For example, the dried biomass can be mechanically extracted to release a crude microbial oil, e.g., using a screw press. See, e.g., PCT Application Publication WO2010120939. Other suitable methods include organic solvent extraction (see, e.g., Frenz et al. 1989, Enzyme Microb. Technol. 11:717); or supercritical CO₂ extraction (see, e.g., Mendes et al. 2003, Inorganica Chimica Acta 356:328-334). Optionally, the crude oil can be refined using standard edible oil refining steps, including degumming, bleaching, and deodorization.

Oils disclosed herein are distinct from other naturally occurring oils that are high in C8:0 and C10:0 medium-chain fatty acids, such as palm oil, palm kernel oil, and coconut oil. For example, levels of contaminants such as carotenoids are far higher in palm oil and palm kernel oil than in the oils described herein. Palm and palm kernel oils in particular contain alpha and beta carotenes and lycopene in much higher amounts than are in the oils described herein. In addition, over 20 different carotenoids are found in palm and palm kernel oil, whereas the oils described herein contain very few carotenoids species and very low levels. In addition, the levels of vitamin E compounds such as tocotrienols are far higher in palm, palm kernel, and coconut oil than in the oils described herein.

Generally, wild-type Prototheca strains have very little or no fatty acids with chain lengths of C8-C14. For example, Prototheca moriformis (UTEX 1435), Prototheca krugani (UTEX 329), Prototheca stagnora (UTEX 1442) and Prototheca zopfii (UTEX 1438) produce no (or undetectable amounts of) C8 fatty acids, between 0-0.01% C10 fatty acids, between 0.03-2.1% C12 fatty acids, and between 1.0-2.1% C14 fatty acids.

In some cases, the oleaginous cells (e.g., Prototheca strains) containing a transgene encoding a variant KASIVa, optionally co-expressing an exogenous lipid biosynthesis enzyme, e.g., a C10-preferring fatty acyl-ACP thioesterase, has a fatty acid profile characterized by at least about 40%, 45%, 50%, 55%, 60%, 65%, 70%, 75%, 80%, 85%, 90%, 95%, 97%, or more, C10 fatty acids. In other cases, the Prototheca strains containing a transgene encoding a variant KASIVa, optionally co-expressing an exogenous lipid biosynthesis enzyme, e.g., a C10-preferring fatty acyl-ACP thioesterase, has activity towards fatty acyl-ACP substrates of chain length C10 and produces fatty acids of the chain length C10.

In some instances, keeping the transgenic Prototheca strains under constant and high selective pressure to retain exogenous genes is advantageous due to the increase in the desired fatty acid of a specific chain length. High levels of exogenous gene retention can also be achieved by inserting exogenous genes into the nuclear chromosomes of the cells using homologous recombination vectors and methods disclosed herein. Recombinant cells containing exogenous genes integrated into nuclear chromosomes are also contemplated.

In some embodiments, oleaginous cells expressing one or more of the polynucleotides described herein can produce an oil with at least 50%, 55%, 60%, 65%, 70%, 75%, 80%, 85%, 90%, or more, C10:0 fatty acids. In some embodiments, oleaginous cells expressing one or more of the polynucleotides described herein can produce an oil with at least 70%, 71%, 72%, 73%, 74%, 75%, 76%, 77%, 78%, 79%, 80%, 81%, 82%, 83%, 84%, 85%, 86%, 87%, 88%, 89%, 90%, or more, C10:0 fatty acids.

In some embodiments, a recombinant cell comprises nucleic acids operable to express an exogenous gene encoding a variant KASIVa that catalyzes the elongation of medium-chain fatty acids from the ACP with a preference for C8-acyl ACP substrates, optionally co-expressing an exogenous lipid biosynthesis enzyme, e.g., a C10-preferring fatty acyl-ACP thioesterase.

In some embodiments, the oil produced has a fatty acid profile that is elevated in C10 fatty acids and reduced in C16:0, C18:0, and C18:1 fatty acids as a result of the expression of the recombinant nucleic acids, preferably as compared to a control cell not expressing said recombinant nucleic acids. In some embodiments, the increase in C10:0 fatty acids is greater than 5%, 10%, 20%, 30%, 40%, 50%, 80%, 100%, 200%, or more, in comparison to an untransformed microalga or a microalga transformed with a wild-type KASIVa. Preferably, the untransformed microalga or the microalga transformed with a wild-type KASIVa is an otherwise identical microalga, e.g. of the same species and preferably the same genetic background. In some embodiments, the increase in C10 fatty acids is at least 2-fold, 3-fold, 4-fold, 5-fold, 6-fold, 7-fold, 8-fold, 9-fold, 10-fold, 11-fold, 12-fold, 13-fold, 14-fold, 15-fold, or more, in comparison to an untransformed microalga or a microalga transformed with a wild-type KASIVa. Preferably, the oil produced by the recombinant cell as defined herein comprises at least about 50%, 55%, 60%, 65%, 70%, 75%, 80%, 85%, 90%, or more, C10:0 fatty acids. The produced oil may further comprise at least one of tridecanoin, MLCT, ergosterol and brassicastol, preferably in an amount as defined herein. Preferably, the produced oil may further comprise at least one of ergosterol and brassicasterol. A preferred MLCT may be selected from the group consisting of CaCaLa triglyceride, CaOCa triglyceride, CaCaP triglyceride and LaLaCa triglyceride, preferably in an amount as described herein.

In some embodiments, the oil produced has a fatty acid profile that is elevated in C10 fatty acids and reduced in C16:0, C18:0, and C18:1 fatty acids as a result of the expression of the KASIVa variant as defined herein, i.e. comprising a glycine at a position corresponding to position 146 of SEQ ID NO: 4, preferably as compared to a control cell not expressing said KASIVa variant. In some embodiments, the increase in C10:0 fatty acids is greater than 5%, 10%, 20%, 30%, 40%, 50%, 80%, 100%, 200%, or more, in comparison to an untransformed microalga or a microalga transformed with a wild-type KASIVa. In some embodiments, the increase in C10 fatty acids is at least 2-fold, 3-fold, 4-fold, 5-fold, 6-fold, 7-fold, 8-fold, 9-fold, 10-fold, 11-fold, 12-fold, 13-fold, 14-fold, 15-fold, or more, in comparison to an untransformed microalga or a microalga transformed with a wild-type KASIVa. Preferably, the oil produced by the recombinant cell as defined herein comprises at least about 50%, 55%, 60%, 65%, 70%, 75%, 80%, 85%, 90%, or more, C10:0 fatty acids. Preferably, the oil produced by the recombinant cell as defined herein comprises at least about 50%, 55%, 60%, 65%, 70%, 75%, 80%, 85%, 90%, or more, C10:0 fatty acids. The produced oil may further comprise at least one of tridecanoin, MLCT, ergosterol and brassicastol, preferably in an amount as defined herein. Preferably, the produced oil may further comprise at least one of ergosterol and brassicasterol. A preferred MLCT may be selected from the group consisting of CaCaLa triglyceride, CaOCa triglyceride, CaCaP triglyceride and LaLaCa triglyceride, preferably in an amount as described herein.

In some embodiments, the oil produced has a fatty acid profile that is elevated in C10 fatty acids and reduced in C16:0, C18:0, and C18:1 fatty acids as a result of the expression of the KASIVa variant as defined herein, i.e. comprising an asparagine at a position corresponding to position 146 of SEQ ID NO: 4, preferably as compared to a control cell not expressing said KASIVa variant. In some embodiments, the increase in C10:0 fatty acids is greater than 5%, 10%, 20%, 30%, 40%, 50%, 80%, 100%, 200%, or more, in comparison to an untransformed microalga or a microalga transformed with a wild-type KASIVa. In some embodiments, the increase in C10 fatty acids is at least 2-fold, 3-fold, 4-fold, 5-fold, 6-fold, 7-fold, 8-fold, 9-fold, 10-fold, 11-fold, 12-fold, 13-fold, 14-fold, 15-fold, or more, in comparison to an untransformed microalga or a microalga transformed with a wild-type KASIVa. Preferably, the oil produced by the recombinant cell as defined herein comprises at least about 50%, 55%, 60%, 65%, 70%, 75%, 80%, 85%, 90%, or more, C10:0 fatty acids. Preferably, the oil produced by the recombinant cell as defined herein comprises at least about 50%, 55%, 60%, 65%, 70%, 75%, 80%, 85%, 90%, or more, C10:0 fatty acids. The produced oil may further comprise at least one of tridecanion, MLCT, ergosterol and brassicastol, preferably in an amount as defined herein. Preferably, the produced oil may further comprise at least one of ergosterol and brassicasterol. A preferred MLCT may be selected from the group consisting of CaCaLa triglyceride, CaOCa triglyceride, CaCaP triglyceride and LaLaCa triglyceride, preferably in an amount as described herein.

In some embodiments, the oil produced has a fatty acid profile that is elevated in C10 fatty acids and reduced in C16:0, C18:0, and C18:1 fatty acids as a result of the expression of the KASIVa variant as defined herein, i.e. comprising a serine at a position corresponding to position 146 of SEQ ID NO: 4, preferably as compared to a control cell not expressing said KASIVa variant. In some embodiments, the increase in C10:0 fatty acids is greater than 5%, 10%, 20%, 30%, 40%, 50%, 80%, 100%, 200%, or more, in comparison to an untransformed microalga or a microalga transformed with a wild-type KASIVa. In some embodiments, the increase in C10 fatty acids is at least 2-fold, 3-fold, 4-fold, 5-fold, 6-fold, 7-fold, 8-fold, 9-fold, 10-fold, 11-fold, 12-fold, 13-fold, 14-fold, 15-fold, or more, in comparison to an untransformed microalga or a microalga transformed with a wild-type KASIVa. Preferably, the oil produced by the recombinant cell as defined herein comprises at least about 50%, 55%, 60%, 65%, 70%, 75%, 80%, 85%, 90%, or more, C10:0 fatty acids. Preferably, the oil produced by the recombinant cell as defined herein comprises at least about 50%, 55%, 60%, 65%, 70%, 75%, 80%, 85%, 90%, or more, C10:0 fatty acids. The produced oil may further comprise at least one of tridecanoin, MLCT, ergosterol and brassicastol, preferably in an amount as defined herein. Preferably, the produced oil may further comprise at least one of ergosterol and brassicasterol. A preferred MLCT may be selected from the group consisting of CaCaLa triglyceride, CaOCa triglyceride, CaCaP triglyceride and LaLaCa triglyceride, preferably in an amount as described herein.

Microalgal oil can also include other constituents produced by the microalgae, or incorporated into the microalgal oil from the culture medium. These other constituents can be present in varying amounts depending on the culture conditions used to culture the microalgae, the species of microalgae, the extraction method used to recover microalgal oil from the biomass and other factors that may affect microalgal oil composition. Non-limiting examples of such constituents include carotenoids, present from 0.1-0.4 micrograms/ml, chlorophyll present from 0-0.02 milligrams/kilogram of oil, gamma tocopherol present from 0.4-0.6 milligrams/100 grams of oil, and total tocotrienols present from 0.2-0.5 milligrams/gram of oil.

The other constituents can include, without limitation, phospholipids, tocopherols, tocotrienols, carotenoids (e.g., alpha-carotene, beta-carotene, lycopene, etc.), xanthophylls (e.g., lutein, zeaxanthin, alpha-cryptoxanthin and beta-crytoxanthin), and various organic or inorganic compounds. Additionally, microalgal oils contain long-chain polyunsaturated fatty acids, particularly eicosapentaenoic acid (EPA).

In some cases, the oil extracted from Prototheca species comprises no more than 0.02 mg/kg chlorophyll. In some cases, the oil extracted from Prototheca species comprises no more than 0.4 mcg/ml total carotenoids. In some cases the Prototheca oil comprises between 0.40-0.60 milligrams of gamma tocopherol per 100 grams of oil. In other cases, the Prototheca oil comprises between 0.2-0.5 milligrams of total tocotrienols per gram of oil.

Oils produced from host cells expressing a variant KASIVa, optionally co-expressing an exogenous lipid biosynthesis enzyme, e.g., a C10-preferring fatty acyl-ACP thioesterase, will have an isotopic profile that distinguishes it, e.g., from blended oils from other sources. The stable carbon isotope value δ13C is an expression of the ratio of 13C/12C relative to a standard (e.g. PDB, carbonite of fossil skeleton of Belemnite americana from Peedee formation of South Carolina). The stable carbon isotope value δ13C (0/00) of the oils can be related to the δ13C value of the feedstock used. In some embodiments the oils are derived from oleaginous organisms heterotrophically grown on sugar derived from a C4 plant such as corn or sugarcane. In some embodiments, the δ13C (0/00) of the oil is from 10 to −17 0/00 or from 13 to −16 0/00.

The oils produced according to the above methods in some cases are made using a microalgal host cell. As described above, the microalga can be, without limitation, fall in the classification of Chlorophyta, Trebouxiophyceae, Chlorellales, Chlorellaceae, or Chlorophyceae. Microalgae of Trebouxiophyceae can be distinguished from vegetable oils based on their sterol profiles. Oil produced by Chlorella protothecoides has been found to contain ergosterol and brassicasterols as major sterols. Both of these sterols feature C24β stereochemistry, in contrast to the C24α stereochemistry found in the majority of common plant sterols. Additional minor sterols present in Chlorella are also believed to primarily have C24β stereochemistry. Thus, the oils produced by the microalgae described above can be distinguished from plant oils by the preponderance of sterols with C24β stereochemistry in the sterols present. For example, the oils produced may contain 22, 23-dihydrobrassicasterol while lacking campesterol; contain clionasterol, while lacking in β-sitosterol, and/or contain poriferasterol while lacking stigmasterol. Alternately, or in addition, the oils may contain significant amounts of Δ⁷-poriferasterol. Accordingly, in some embodiments, the oils produced according to the methods described herein lack C24-α sterols.

In one embodiment, the oils provided herein are not vegetable oils. Vegetable oils are oils extracted from plants and plant seeds. Vegetable oils can be distinguished from the non-plant oils provided herein on the basis of their oil content. A variety of methods for analyzing the oil content can be employed to determine the source of the oil or whether adulteration of an oil provided herein with an oil of a different (e.g. plant) origin has occurred. The determination can be made on the basis of one or a combination of the analytical methods. These tests include but are not limited to analysis of one or more of free fatty acids, fatty acid profile, total triacylglycerol content, diacylglycerol content, peroxide values, spectroscopic properties (e.g. UV absorption), sterol profile, sterol degradation products, antioxidants (e.g. tocopherols), pigments (e.g. chlorophyll), d13C values and sensory analysis (e.g. taste, odor, and mouth feel). Many such tests have been standardized for commercial oils such as the Codex Alimentarius standards for edible fats and oils.

Sterol profile analysis is a particularly well-known method for determining the biological source of organic matter. Campesterol, β-sitosterol, and stigamsterol are common plant sterols, with β-sitosterol being a principle plant sterol. For example, β-sitosterol was found to be in greatest abundance in an analysis of certain seed oils, approximately 64% in corn, 29% in rapeseed, 64% in sunflower, 74% in cottonseed, 26% in soybean, and 79% in olive oil (Gul et al. J. Cell and Molecular Biology 5:71-79, 2006).

Oil isolated from Prototheca moriformis strain UTEX1435 was separately clarified (CL), refined and bleached (RB), or refined, bleached and deodorized (RBD) and was tested for sterol content according to the procedure described in JAOCS vol. 60, no. 8, August 1983. Results of the analysis are shown below (units in mg/100 g) in Table C:

TABLE C

| | | | | | Refined, |
|---|---|---|---|---|---|
| | | | | Refined & | bleached, & |
| | Sterol | Crude | Clarified | bleached | deodorized |
| 1 | Ergosterol | 384 | 398 | 293 | 302 |
| | | (56%) | (55%) | (50%) | (50%) |
| 2 | 5,22-cholestadien- | 15 | 19 | 14 | 15 |
| | 24-methyl-3-ol | (2.1%) | (2.6%) | (2.4%) | (2.5%) |
| | (Brassicasterol) | | | | |
| 3 | Other sterols | 287 | 302 | 283 | 284 |
| | Total sterols | 686 | 719 | 590 | 601 |

These results show three striking features. First, ergosterol was found to be the most abundant of all the sterols, accounting for about 50% or more of the total sterols. Ergosterol is a sterol commonly found in fungus and not commonly found in plants, and its presence particularly in significant amounts serves as a useful marker for non-plant oils. Secondly, the oil was found to contain brassicasterol. In summary, Prototheca moriformis strain UTEX1435 has been found to contain both significant amounts of ergosterol and only trace amounts of β-sitosterol as a percentage of total sterol content. Accordingly, the ratio of ergosterol: β-sitosterol or in combination with the presence of brassicasterol can be used to distinguish this oil from plant oils.

In some embodiments, the oil content of an oil provided herein contains, as a percentage of total sterols, less than 20%, 15%, 10%, 5%, 4%, 3%, 2%, or 1% β-sitosterol. In other embodiments the oil is free from β-sitosterol.

In some embodiments, the oil is free from one or more of β-sitosterol, campesterol, or stigmasterol. In some embodiments the oil is free from β-sitosterol, campesterol, and stigmasterol. In some embodiments the oil is free from campesterol. In some embodiments the oil is free from stigmasterol.

In some embodiments, the oil content of an oil provided herein contains ergosterol or brassicasterol or a combination of the two. In some embodiments, the oil content contains, as a percentage of total sterols, at least 5%, 10%, 20%, 25%, 35%, 40%, 45%, 50%, 55%, 60%, or 65% ergosterol. In some embodiments, the oil content contains, as a percentage of total sterols, at least 25% ergosterol. In some embodiments, the oil content contains, as a percentage of total sterols, at least 40% ergosterol. In some embodiments most abundant sterol is ergosterol. In some embodiments, the oil content contains, as a percentage of total sterols, at least 5%, 10%, 20%, 25%, 35%, 40%, 45%, 50%, 55%, 60%, or 65% of a combination of ergosterol and brassicasterol.

In some embodiments, the oil content contains, as a percentage of total sterols, at least 1%, 2%, 3%, 4% or 5% brassicasterol. In some embodiments, the oil content contains, as a percentage of total sterols less than 10%, 9%, 8%, 7%, 6%, or 5% brassicasterol.

In some embodiments the ratio of ergosterol to brassicasterol is at least 5:1, 10:1, 15:1, or 20:1.

Sterols contain from 27 to 29 carbon atoms (C27 to C29) and are found in all eukaryotes. Animals exclusively make C27 sterols as they lack the ability to further modify the C27 sterols to produce C28 and C29 sterols. Plants however are able to synthesize C28 and C29 sterols, and C28/C29 plant sterols are often referred to as phytosterols. The sterol profile of a given plant is high in C29 sterols, and the primary sterols in plants are typically the C29 sterols β-sitosterol and stigmasterol. In contrast, the sterol profile of non-plant organisms contain greater percentages of C27 and C28 sterols. For example the sterols in fungi and in many microalgae are principally C28 sterols. The sterol profile and particularly the striking predominance of C29 sterols over C28 sterols in plants has been exploited for determining the proportion of plant and marine matter in soil samples (Huang, Wen-Yen, Meinschein W. G., “Sterols as ecological indicators”; Geochimica et Cosmochimia Acta. Vol 43. pp 739-745).

In some embodiments the primary sterols in the microalgal oils provided herein are sterols other than β-sitosterol and stigmasterol. In some embodiments of the microalgal oils, C29 sterols make up less than 50%, 40%, 30%, 20%, 10%, or 5% by weight of the total sterol content.

In some embodiments the microalgal oils provided herein contain C28 sterols in excess of C29 sterols. In some embodiments of the microalgal oils, C28 sterols make up greater than 50%, 60%, 70%, 80%, 90%, or 95% by weight of the total sterol content. In some embodiments the C28 sterol is ergosterol. In some embodiments the C28 sterol is brassicasterol.

The oils provided herein have many applications. Certain foods which contain medium chain triglycerides (MCT) are known to provide an instance energy source. Because the shorter chain length of the MCT fatty acids are rapidly broken down, they are known to be rapidly absorbed into the body. Because the calories contained in MCTs are more efficiently turned into energy and used by the body, they are less likely stored as fat, potentially aiding in weight loss. In a recent study, ketogenic MCTs were shown to increase brain energy metabolism in Alzheimer's disease. See J. Alzheimer's Disease 64 (2018:551-561). Therefore, the MCTs in the oils provided herein may be potentially useful in providing alternative energy source, and in treating or enhancing various neurological conditions.

The oils provided herein also contain a significant amount of medium-long-chain triglycerides (MLCT). Certain MLCTs were shown to be effective in decreasing the accumulation of body fat in animals and humans. MLCTs are also shown to have health benefits in targeting specific disease and metabolic conditions. Further, MLCTs can be incorporated in the mainstream foods to substitute soybean oil or palm olein in salad dressing formulation and frying oil, respectively. See Koh et al., International Food Research Journal 18:355-366 (2011).

The medium-chain fatty acids derived from hydrolysis of these oils may be particularly useful in food, fuel and oleochemical applications including the production of lubricants and surfactants. For example, fatty acids derived from the cells can be esterified, cracked, reduced to an aldehyde or alcohol, aminated, sulfated, sulfonated, or subjected to other chemical process known in the art.

The following examples, which are offered to illustrate, but not to limit, the compositions and methods described herein.

### EXAMPLES

The following examples are offered to illustrate, but not to limit the claimed invention.

### Example 1. Production of an Oil Enriched in C10:0 Fatty Acids

A method to produce an oil enriched in fatty acids with a 10-carbon chain length (C10 fatty acids) in P. moriformis is to introduce a β-ketoacyl-ACP synthase (KAS) gene and a fatty acyl-ACP thioesterase (FAT) gene from an oilseed plant known to make C10 fatty acids, such as Cuphea paucipetala, into the algal genome. Here, we report on the ability to produce an oil with >70% C10:0 fatty acids using the transgenic P. moriformis strain S9109, which is derived from the non-recombinant base strain S7485 and expresses two copies of the C. paucipetala FATB1Δ28 (CpauFATB1Δ28, SEQ ID NO: 1) thioesterase gene variant and three copies of the wild-type C. paucipetala KASIVa (CpauKASIVa, SEQ ID NO: 1, 2) gene through two successive transformations. S7485 is a classically-improved derivative of the wild-type strain UTEX 1435, which was obtained from the University of Texas culture collection and classically mutagenized to increase oil yield and productivity. The classical mutagenesis did not substantively alter the fatty acid profile of the oil produced by S7485 when compared to UTEX 1435. Neither S7485 nor UTEX 1435 produces an oil with a detectable amount of C10:0 fatty acids.

The expression construct pSZ5767 (SEQ ID NO: 1) was initially transformed into strain S7485 to introduce one copy of CpauFATB1Δ28 and one copy of CpauKASIVa into each allele of the THI4 locus through a double integration event (total of two copies of each heterologous gene in the algal genome) to generate the intermediate strain S8714. Construct pSZ5767 also contains an expression cassette for the Saccharomyces cerevisiae SUC2 (ScSUC2) gene, which enables the transformed cells to grow on media with sucrose as the sole carbon source. The expression construct pSZ6156 (SEQ ID NO: 2) was then transformed into strain S8714 (a clonally-purified isolate) to introduce a third copy of CpauKASIVa to the algal geneome at the DAO1b locus to generate the final strain S9109. In addition to the CpauKASIVa gene, construct pSZ6156 also contains an expression cassette for the Arabidopsis thaliana THIC^{L337M} gene variant (AtTHIC^{L337M}) which is used for the selection of transformed cells on medium without thiamine supplementation. Expression of the CpauFATB1Δ28 and CpauKASIVa genes in the transgenic strains is driven by the constitutive PmSAD2 promoter.

To obtain sufficient oil for fatty acid and TAG profile analyses, strain 9109 (a clonally-purified isolate) was cultured under low nitrogen conditions at pH 5 and 28° C. in 250-mL baffled flasks for 5 days with an 8% (v v) inoculum. Each flask contained 46 mL of lipid production medium that comprised 60 g/L sucrose as the sole carbon source. After 3 days of fermentation, an additional 10 g/L of sucrose was added to each production culture. Other details pertaining to the seed train, media composition, and culture conditions are described in the Examples of PCT Patent Application WO 2018/067849.

Cells from the production cultures were recovered by centrifugation and dried by lyophilization for analysis for fatty acid profile and lipid (as glycerides) titer. Ten to forty milligrams of lyophilized biomass were resuspended in 2 mL of 5% (v v) H₂SO₄ in methanol, and 200 L of toluene containing an appropriate amount of a suitable internal standard (C19:0) were added. The resulting mixture was sonicated briefly to disperse the biomass, and then heated at 70-75° C. for 3.5 hours with intermittent sonicating and vortex mixing. Heptane (2 mL) was added for the extraction of the fatty acid methyl esters, followed by addition of 2 mL of 10% (w/v) K₃PO₄ (aq) to neutralize. The mixture was agitated vigorously, and a portion of the upper layer was transferred to a vial containing Na₂SO₄ (anhydrous) for gas chromatography analysis using standard FAME GC/FID (fatty acid methyl ester gas chromatography flame ionization detection) methods.

Triacylglycerols were identified by LC/MS analysis using a Shimadzu Nexera ultra high performance liquid chromatography system that included a SIL-30AC autosampler, two LC-30AD pumps, and a DGU-20A5 in-line degasser, coupled to a Shimadzu LCMS 8030 triple quadrupole mass spectrometer equipped with an APCI source. Data was acquired using a Q3 scan of m z 300-950 at a scan speed of 1363 u/sec in positive ion mode with the CID gas (argon) pressure set to 230 kPa. The APCI, desolvation line, and heat block temperatures were set to 300, 250, and 200° C., respectively, the flow rates of the nebulizing and drying gases were 3.0 L/min and 5.0 L/min, respectively, and the interface voltage was 4500 V. Oil samples were dissolved in dichloromethane-methanol (1:1) to a concentration of 5 mg/mL, and 0.4 L of sample was injected onto an Thermo Scientific Acclaim RSLC 120 C18 (2.2 m, 2.1×250 mm) column maintained at 15° C. in a Cole-Parmer column heater/chiller. A linear gradient from 30% dichloromethane-2-propanol (1:1)/acetonitrile to 56% dichloromethane-2-propanol (1:1)/acetonitrile over 34 minutes at 0.48 mL/min was used for chromatographic separations.

Triacylglycerol profiles were generated by HPLC with RID (Refractive Index Detector) using AOCS method Ce 5c-93, modified to include two columns. Hichrom Alltima HP C18-HL and Acclaim 120 C18 columns, both 5 m, 4.6×250 mm, in series, were used.

As shown in Table 1, strain S9109 is capable of producing an oil with a high level of C10:0 fatty acids, which account for 72% of the total fatty acids. At the same time, 46% of the triglycerides produced are medium-chain fatty acid triglycerides (MCTs), in which all three fatty acids bound to the glycerol backbone are medium-chain fatty acids having 6 to 10 carbons (C6-C10) (Table 2). Tridecanoin (CaCaCa), which comprise 45% of the total triglycerides, is the major MCT species. In addition, 51% of the triglycerides produced are also medium and long chain fatty acid triglycerides (MLCTs), in which at least one medium chain fatty acid having 6 to 10 carbons (C6-C10) and at least one long chain fatty acid having 12 carbons or more are bound to the glycerol backbone. These results demonstrate that an oil rich in C10:0 fatty acids, MCTs, and MLCTs can be produced through the introduction of two heterologous genes from the oilseed plant C. paucipetala into the oleaginous microalga P. moriformis.

TABLE 1
Fatty acid profile of oil extracted from dried biomass of strain S9109.*

| | Fatty Acid Profile (%) | | | | | | | | |
|---|---|---|---|---|---|---|---|---|---|
| | | | | | | C14:1 | | C16:1 | |
| Sample | C8:0 | C10:0 | C11:0 | C12:0 | C14:0 | cis-9 | C16:0 | cis-9 | C17:0 |
| S9109 dried | 0.5 | 72.0 | 0.1 | 6.8 | 2.6 | 0.0 | 5.4 | 0.1 | 0.0 |
| biomass | | | | | | | | | |

| | Fatty Acid Profile (%) | | | | | | | |
|---|---|---|---|---|---|---|---|---|
| Sample | C18:0 | C18:1 | C18:2 | C18:3α | C20:0 | C20:1 | C21:0 | C22:0 |
| S9109 dried | 0.5 | 9.0 | 2.5 | 0.3 | 0.1 | 0.0 | 0.1 | 0.0 |
| biomass | | | | | | | | |

Strain S9109 was cultured in 250-mL baffled flasks under low-nitrogen conditions at pH 5 and 28° C. for 5 days. Results are presented as average values from replicate analyses.

TABLE 2
Non-regiospecific TAG profile of oil extracted from
dried biomass of strain S9109.*

| TAG | % of |
|---|---|
| (non-regiospecific) | Triglycerides |
| CyCaCa | 1.5 |
| CaCaCa | 44.8 |
| CaLnCa | 0.3 |
| CaCaLa | 12.6 |
| CaLCa | 3.4 |
| CaCaM + LaLaCa | 5.5 |
| CaLLa | 0.4 |
| CaOCa | 12.5 |
| CaCaP | 8.3 |
| CaOLa | 3.0 |
| CaOL | 0.3 |
| CaLP | 1.6 |
| CaOM | 0.5 |
| CaOO | 0.8 |
| CaOP | 2.0 |
| Total TAGs identified: | 97.4 |

Strain S9109 was cultured in 250-mL baffled flasks under low-nitrogen conditions at pH 5 and 28° C. for 5 days. Cy = caprylate (C8:0), Ca = caprate (C10:0), La = laurate (C12:0), M = myristate (C14:0), P = palmitate (C16:0), O = oleate (C18:1), L = linoleate (C18:2), and Ln = α-linolenate (C18:3 α).

### Example 2. Improved Variant of Cuphea paucipetala KASIVa Enzyme

To minimize the number of copies of the CpauKASIVa gene needed to produce an oil with >70% C10:0 fatty acids in P. moriformis, the heterologous KAS enzyme was subjected to site-directed mutagenesis using known techniques familiar to those skilled in the art to increase its activity. Here, we report on the improvement in C10 activity achieved by replacing the native threonine residue at position 146 of the C. paucipetala KASIVa (CpauKASIVa) with a serine residue. Results obtained from expression of the wild-type CpauKASIVa (SEQ NO ID: 3) or the improved CpauKASIVa ^{T146S} variant with the Thr146 to Ser mutation (SEQ NO ID: 4) in conjunction with the C. paucipetala FATB1Δ28 thioesterase variant (CpauFATB1Δ28, SEQ NO ID: 5) for C10 production in P. moriformis are also described.

An expression construct that targets integration of the CpauKASIVa^{T146S} gene variant (pSZ6871, SEQ ID NO: 6) or the wild-type CpauKASIVa gene (pSZ6757, SEQ ID NO: 7) to the THI4 locus in P. moriformis was transformed into the host strain S9189, which already harbors two copies of the CpauFATB1Δ28 thioesterase gene, one at each allele of its DAO1b locus. Constructs pSZ6757 and pSZ6871 also contain an expression cassette for the S. cerevisiae SUC2 (ScSUC2) gene, which enables growth of the resulting transformants on medium containing sucrose as the sole carbon source. Strain S9189 is a clonally-purified transformant that is stable in phenotype and was obtained through transformation of the expression construct pSZ6712 (SEQ ID NO: 8) into the base strain S9112, which itself does not produce oil with C10:0 fatty acids. In addition to the CpauFATB1Δ28 gene, construct pSZ6712 also contains an expression cassette for the Arabidopsis thaliana THIC^{L337M} gene variant, which is used as a selectable marker for the growth of transformants on medium without thiamine supplementation. The expression of CpauKASIVa, CpauKASIVa^{T146S}, and CpauFATB1Δ28 in the transgenic strains is driven by the pH 7-inducible PmAMT3 promoter.

Clonally-purified transformants derived from strain S9189 and expressing the CpauKASIVa^{T146S} gene variant (pSZ6871) or wild-type CpauKASIVa gene (pSZ6757) were evaluated in 3-day lipid production cultures at pH 7 (as detailed in the Examples of PCT Patent Application WO 2018/067849) to determine their fatty acid profiles. The parental strain S9189 and the base strain S9112 were also assessed under the same conditions as controls. As presented in Table 3, single and double integrants of the wild-type CpauKASIVa gene (pSZ6757) can produce oils with average C10:0 levels of 56% and 76%, respectively. In comparison, single and double integrants of the CpauKASIVa^{T146S} gene variant (pSZ6871) yielded oils with higher C10:0 levels, which reached 59% and 79%, respectively. These results also indicate that the expression of a second copy of the KAS transgene can significantly boost the C10:0 level of the oils. In contrast, the parental strain S9189, which harbors two copies of the CpauFATB1Δ28 thioesterase gene but does not express either one of the KAS transgenes, produced oils with only approximately 21% C10:0. No C10:0 fatty acids were detected in the oil accumulated by the base strain S9112.

TABLE 3

| | | | | Average | | | |
|---|---|---|---|---|---|---|---|
| Strain/Transformants | CpauKASIVa | Integration | n | C8:0 (%) | C10:0 (%) | C12:0 (%) | C14:0 (%) |
| S9189 - pSZ6871 | T146S | Single | 8 | 0.6 | 58.3 | 6.1 | 4.2 |
| S9189 - pSZ6871 | T146S | Double | 12 | 0.9 | 78.9 | 5.6 | 2.3 |
| S9189 - pSZ6757 | Wild-type | Single | 6 | 0.6 | 54.9 | 5.8 | 4.6 |
| S9189 - pSZ6757 | Wild-type | Double | 13 | 0.9 | 75.9 | 5.9 | 2.7 |
| S9189 (parent) | — | — | 2 | 0.2 | 21.2 | 2.8 | 4.2 |
| S9112 (base strain) | — | — | 2 | 0.1 | 0.0 | 0.1 | 2.3 |

The parental strain S9189 was transformed with construct pSZ6871 (CpauKASIVa^{T146S}) or construct pSZ6757 (wild-type CpauKASIVa). Clonally-purified transformants were tested in three-day lipid production cultures at pH 7 under low nitrogen conditions. Day-3 fatty acid profiles are shown for both single and double integrants of the heterologous KAS genes. n = total of replicates or transformants tested.

### Example 3. Saturation Mutagenesis at Amino Acid Residue 146 of the Cuphea paucipetala KASIVa Enzyme

Example 2 demonstrated that a Thr to Ser mutation at amino acid residue 146 of the CpauKASIVa enzyme (SEQ NO ID: 4) can increase the C10:0 fatty acid level of the oil produced by P. moriformis when co-expressed with a thioesterase (CpauFATB1Δ28, SEQ NO ID: 5) enzyme. In this example, results from saturation mutagenesis at position 146 of CpauKASIVa indicating the importance of that amino acid position in producing oils with elevated C10:0 fatty acids are described.

An expression construct that targets integration of the wild-type CpauKASIVa gene (pSZ6756, SEQ NO ID: 13), the CpauKASIVa^{T146S} gene variant (pSZ6921, SEQ NO ID: 14), or the remaining eighteen CpauKASIVa^{T146X} gene variants (pSZ7123-pSZ7140, SEQ NO ID: 14) to the THI4 locus in P. moriformis was transformed into the host strain S9316, which harbors two copies of the CpauFATB1Δ28 thioesterase gene, one at each allele of its DAO1 locus. These constructs also contain an expression cassette for the S. cerevisiae SUC2 (ScSUC2) gene, which enables growth of the resulting transformants on medium containing sucrose as the sole carbon source. Similar to strain S9189 in Example 2, strain S9316 is a clonally-purified transformant that is stable in phenotype and was obtained through transformation of the expression construct pSZ6769 (SEQ NO ID: 15) into the base strain S9281, which itself does not produce oil with C10:0 fatty acids. In addition to the CpauFATB1Δ28 gene, construct pSZ6769 also contains an expression cassette for the A. thaliana THIC^{L337M} gene variant, which is used as a selectable marker for the growth of transformants on medium without thiamine supplementation. The expression of the CpauKASIVa^{T146X} variants and CpauFATB1Δ28 in the transgenic strains is driven by the constitutive PmSAD2-1 promoter.

Clonally-purified transformants derived from strain S9316 and expressing a single copy of one of the CpauKASIVa^{T146X} variants (pSZ6756, pSZ6921, or pSZ7123-pSZ7140) were evaluated in 5-day lipid production cultures at pH 7 (as detailed in the Examples of PCT Patent Application WO2018/067849) to determine their fatty acid profiles. The parental strain S9316 and a base strain, S7485, which itself does not produce oil with C10:0 fatty acids, were also assessed under the same conditions as controls. The average C10:0 fatty acid levels of the oils produced by these transformants and strains are presented in Table 4. While the parental strain S9316 can produce an oil with 19% C10:0, the addition of one copy of the wild-type CpauKASIVa (pSZ6756) enabled the resulting transformants to reach 44% C10:0. Expression of the T146S variant (pSZ6921) instead of the wild-type CpauKASIVa further elevated the C10:0 level to 52%. This was the highest C10:0 level observed across all twenty amino acid substitutions. Replacement of Thr146 with one of the other eighteen amino acids yielded C10:0 levels that ranged from 25% to 50%. Although the C10:0 levels (48-50%) achieved with the T146G and T146N variants (pSZ7123 and pSZ7130) were not as high as with the T146S variant, they still exceeded the C10:0 level achieved with the wild-type enzyme. The sensitivity of the C10:0 level to the choice of amino acid at position 146 of the CpauKASIVa enzyme demonstrates the importance of this amino acid position on the biosynthesis of triglycerides rich in C10:0 fatty acids.

TABLE 4

| | | | Average | | | |
|---|---|---|---|---|---|---|
| Strain/ | | | C8:0 | C10:0 | C12:0 | C14:0 |
| Transformants | CpauKASIVa | n | (%) | (%) | (%) | (%) |
| S9316 - pSZ7123 | T146G | 8 | 0.3 | 49.5 | 6.2 | 4.7 |
| S9316 - pSZ7124 | T146E | 8 | 0.2 | 33.4 | 4.5 | 5.2 |
| S9316 - pSZ7125 | T146D | 7 | 0.2 | 31.9 | 4.3 | 5.1 |
| S9316 - pSZ7126 | T146V | 8 | 0.2 | 27.6 | 3.7 | 4.8 |
| S9316 - pSZ7127 | T146A | 7 | 0.3 | 39.3 | 5.1 | 5.1 |
| S9316 - pSZ7128 | T146R | 5 | 0.3 | 42.1 | 5.5 | 5.2 |
| S9316 - pSZ7129 | T146K | 7 | 0.3 | 38.2 | 5.0 | 5.2 |
| S9316 - pSZ7130 | T146N | 8 | 0.3 | 47.9 | 6.0 | 4.8 |
| S9316 - pSZ7131 | T146M | 8 | 0.2 | 32.5 | 4.4 | 5.0 |
| S9316 - pSZ7132 | T146I | 8 | 0.2 | 24.9 | 3.4 | 4.6 |
| S9316 - pSZ7133 | T146W | 8 | 0.2 | 26.2 | 3.6 | 4.7 |
| S9316 - pSZ7134 | T146C | 7 | 0.3 | 40.1 | 5.3 | 5.2 |
| S9316 - pSZ7135 | T146Y | 8 | 0.2 | 31.7 | 4.3 | 5.1 |
| S9316 - pSZ7136 | T146L | 7 | 0.2 | 32.9 | 4.4 | 5.1 |
| S9316 - pSZ7137 | T146F | 8 | 0.2 | 30.6 | 4.1 | 5.0 |
| S9316 - pSZ7138 | T146Q | 8 | 0.3 | 44.3 | 5.6 | 5.1 |
| S9316 - pSZ7139 | T146H | 8 | 0.3 | 39.0 | 5.1 | 5.0 |
| S9316 - pSZ7140 | T146P | 8 | 0.2 | 25.7 | 3.4 | 4.5 |
| S9316 - pSZ6756 | WT | 7 | 0.3 | 44.3 | 5.7 | 5.0 |
| S9316 - pSZ6921 | T146S | 7 | 0.4 | 51.5 | 6.3 | 4.6 |
| S9316 (parent) | — | 3 | 0.1 | 19.1 | 2.7 | 3.9 |
| S7485 (base strain) | — | 3 | 0.0 | 0.0 | 0.1 | 2.1 |

The parental strain S9316 was transformed with construct pSZ6756, pSZ6921, or pSZ7123-pSZ7140. Each of these constructs expresses either the wild-type CpauKASIVa or one of the enzyme variants containing an amino acid substitution at position 146. Clonally-purified transformants were tested in five-day lipid production cultures at pH 7 under low nitrogen conditions. The caprylic, capric, lauric, and myristic fatty acid levels of the oils produced by single integrants of the heterologous KAS genes are shown. The variants highlighted and in bold represent the amino acid substitutions that conferred higher C10.0 levels. n=total of replicates or transformants tested.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A microbial oil comprising triglycerides, wherein the oil comprises at least about 35% tridecanoin.

2. The microbial oil of claim 1, wherein the oil comprises at least about 44% tridecanoin.

3. The microbial oil of claim 1, wherein the oil comprises about 40% to about 50% of tridecanoin.

4. The microbial oil of claim 1, wherein the oil further comprises at least about 40% of medium- and long-chain triglycerides (MLCT).

5. The microbial oil of claim 4, wherein the oil comprises about 40% to about 60% of the MLCT.

6. The microbial oil of claim 4, wherein about at least about 5% of the MLCT in the oil is caprate-caprate-laurate (CaCaLa) triglyceride.

7. The microbial oil of claim 4, wherein at least about 5% of the MLCT is caprate-oleate-caprate (CaOCa) triglyceride.

8. The microbial oil of claim 4, wherein at least about 5% of the MLCT is caprate-caprate-palmitate (CaCaP) triglyceride.

9. The microbial oil of claim 4, wherein at least about 3% of the MLCT is a combination of caprate-caprate-myristate (CaCaM) triglyceride and laurate-laurate-caprate (LaLaCa) triglyceride.

10. The microbial oil of claim 1, wherein the C10:0 fatty acid content of the oil is at least about 45% of total fatty acids derivable from the oil.

11. The microbial oil of claim 1, wherein the C10:0 fatty acid content of the oil is about 40% to about 90% of total fatty acids derivable from the oil.

12. The microbial oil of claim 1, wherein the C8:0 fatty acid content of the oil is present in an amount less than about 5% of total fatty acids derivable from the oil.

13. The microbial oil of claim 1, wherein the C12:0 fatty acid content of the oil is present in an amount less than 15% of the total fatty acids derivable from the oil.

14. The microbial oil of claim 1, wherein the C14:0 fatty acid content of the oil is present in an amount less than about 15% of the total fatty acids derivable from the oil.

15. The microbial oil of claim 1, wherein the oil is obtained from an oleaginous microbial cell.

16. The microbial oil of claim 1, wherein the oil is obtained from an oleaginous microalga.

17. The microbial oil of claim 16, wherein the oleaginous microalga is of the genus Prototheca.

18. The microbial oil of claim 17, wherein the oleaginous microalga is Prototheca moriformis.

19. The microbial oil of claim 1, wherein the oil further comprises ergosterol and/or brassicasterol.

20. The microbial oil of claim 19, wherein the ratio of ergosterol to brassicasterol in the oil is at least 5:1.


SEQUENCE LISTING (25 sequences)

SEQ ID NO: 1 - DNA, 12642 bp, synthetic construct
  misc_feature 1..12642: note = synthetic
  source 1..12642: mol_type = other DNA
agcggaagagcgcccaatgtttaaacccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggaattcctgaagaatgggaggcaggtgttgttgattatgagtgtgtaaaagaaaggggtagagagccgtcctcagatccgactactatgcaggtagccgctcgcccatgcccgcctggctgaatattgatgcatgcccatcaaggcaggcaggcatttctgtgcacgcaccaagcccacaatcttccacaacacacagcatgtaccaacgcacgcgtaaaagttggggtgctgccagtgcgtcatgccaggcatgatgtgctcctgcacatccgccatgatctcctccatcgtctcgggtgtttccggcgcctggtccgggagccgttccgccagatacccagacgccacctccgacctcacggggtacttttcgagcgtctgccggtagtcgacgatcgcgtccaccatggagtagccgaggcgccggaactggcgtgacggagggaggagagggaggagagagaggggggggggggggggggatgattacacgccagtctcacaacgcatgcaagacccgtttgattatgagtacaatcatgcactactagatggatgagcgccaggcataaggcacaccgacgttgatggcatgagcaactcccgcatcatatttcctattgtcctcacgccaagccggtcaccatccgcatgctcatattacagcgcacgcaccgcttcgtgatccaccgggtgaacgtagtcctcgacggaaacatctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacacatatggcttccgcggcattcaccatgtcggcgtgccccgcgatgactggcagggcccctggggcacgtcgctccggacggccagtcgccacccgcctgaggggctccaccttccagtgcctggtgaactcccacatcgacccctgcaaccagaacgtgtcctccgcctccctgtccttcctgggcgacaacggcttcggctccaaccccttccgctccaaccgcggccaccgccgcctgggccgcgcctcccactccggcgaggccatggccgtggccctgcagcccgcccaggaggtggccaccaagaagaagcccgccatcaagcagcgccgcgtggtggtgaccggcatgggcgtggtgacccccctgggccacgagcccgacgtgttctacaacaacctgctggacggcgtgtccggcatctccgagatcgagaccttcgactgcacccagttccccacccgcatcgccggcgagatcaagtccttctccaccgacggctgggtggcccccaagctgtccaagcgcatggacaagttcatgctgtacctgctgaccgccggcaagaaggccctggccgacgccggcatcaccgaggacgtgatgaaggagctggacaagcgcaagtgcggcgtgctgatcggctccggcatgggcggcatgaagctgttcaacgactccatcgaggccctgcgcgtgtcctacaagaagatgaaccccttctgcgtgcccttcgccaccaccaacatgggctccgccatgctggccatggacctgggctggatgggccccaactactccatctccaccgcctgcgccacctccaacttctgcatcctgaacgccgccaaccacatcatccgcggcgaggccgacatgatgctgtgcggcggctccgacgccgtgatcatccccatcggcctgggcggcttcgtggcctgccgcgccctgtcccagcgcaactccgaccccaccaaggcctcccgcccctgggactccaaccgcgacggcttcgtgatgggcgagggcgccggcgtgctgctgctggaggagctggagcacgccaagaagcgcggcgccaccatctacgccgagttcctgggcggctccttcacctgcgacgcctaccacatgaccgagccccaccccgacggcgccggcgtgatcctgtgcatcgagaaggccctggcccagtccggcgtgtcccgcgaggacgtgaactacatcaacgcccacgccacctccacccccgccggcgacatcaaggagtaccaggccctggcccactgcttcggccagaactccgagctgcgcgtgaactccaccaagtccatgatcggccacctgctgggcgccgccggcggcgtggaggccgtgaccgtgatccaggccatccgcaccggctggatccaccccaacctgaacctggaggaccccgacgaggccgtggacgccaagttcctggtgggccccaagaaggagcgcctgaacgtgaaggtgggcctgtccaactccttcggcttcggcggccacaactcctccatcctgttcgccccctacaacaccatgtacccctacgacgtgcccgactacgcctgatatcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgggagcagttgtcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtaacaatggccaccgcctccaccttctccgccttcaacgcccgctgcggcgacctgcgccgctccgccggctccggcccccgccgccccgcccgccccctgcccgtgcgcgccgccatcaacgcctccgcccaccccaaggccaacggctccgccgtgaacctgaagtccggctccctgaacacccaggaggacacctcctcctccccccccccccgcgccttcctgaaccagctgcccgactggtccatgctggtggactccgtgggcctgaagtccgtggtgctggacggcctggtgtcccgccagatcttctccatccgctcctacgagatcggcgccgaccgcaccgcctccatcgagaccctgatgaaccacctgcaggagacctccatcaaccactgcaagtccctgggcctgctgaacgacggcttcggccgcacccccggcatgtgcaagaacgacctgatctgggtgctgaccaagatgcagatcatggtgaaccgctaccccacctggggcgacaccgtggagatcaacacctggttctcccactccggcaagatcggcatggcctccgactggctgatcaccgactgcaacaccggcgagatcctgatccgcgccacctccgtgtgggccatgatgaaccagaagacccgccgcttctcccgcctgccctacgaggtgcgccaggagctgaccccccactacgtggactccccccacgtgatcgaggacaacgaccgcaagctgcacaagttcgacgtgaagaccggcgactccatccgcaagggcctgaccccccgctggaacgacctggacgtgaaccagcacgtgtccaacgtgaagtacatcggctggatcctggagtccatgcccatcgaggtgctggagacccaggagctgtgctccctgaccgtggagtaccgccgcgagtgcggcatggactccgtgctggagtccgtgaccgccatggacccctccgaggacgagggccgctcccagtacaagcacctgctgcgcctggaggacggcaccgacatcgtgaagggccgcaccgagtggcgccccaagaacgccggcaccaacggcgccatctccaccgccaagccctccaacggcaactccgtgtccatggactacaaggaccacgacggcgactacaaggaccacgacatcgactacaaggacgacgacgacaagtgactcgagggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgactgaagagcgtttaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggcaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagga

SEQ ID NO: 2 - DNA, 9742 bp, synthetic construct
  misc_feature 1..9742: note = synthetic
  source 1..9742: mol_type = other DNA
agcggaagagcgcccaatgtttaaacagcccgcaccctcgttgatctgggagccctgcgcagccccttaaatcatctcagtcaggtttctgtgttcaactgagcctaaagggctttcgtcatgcgcacgagcacacgtatatcggccacgcagtttctcaaaagcggtagaacagttcgcgagccctcgtaggtcgaaaacttgcgccagtactattaaattaaattaattgatcgaacgagacgcgaaacttttgcagaatgccaccgagtttgcccagagaatgggagtggcgccattcaccatccgcctgtgcccggcttgattcgccgagacgatggacggcgagaccagggagcggcttgcgagccccgagccggtagcaggaacaatgatcgacaatcttcctgtccaattactggcaaccattagaaagagccggagcgcgttgaaagtctgcaatcgagtaatttttcgatacgtcgggcctgctgaaccctaaggctccggactttgtttaaggcgatccaagatgcacgcggccccaggcacgtatctcaagcacaaaccccagccttagtttcgagactttgggagatagcgaccgatatctagtttggcattttgtatattaattacctcaagcaatggagcgctctgatgcggtgcagcgtcggctgcagcacctggcagtggcgctagggtcgccctatcgctcggaacctggtcagctggctcccgcctcctgctcagcctcttccggtaccgtaatcccgaggttggccccgcttccgctggacacccatcgcatcttccggctcgcccgctgtcgagcaagcgccctcgtgcgcgcaacccttgtggtgcctgcccgcagagccgggcataaaggcgagcaccacacccgaaccagtccaatttgctttctgcattcactcaccaacttttacatccacacatcgtactaccacacctgcccagtcgggtttgatttctattgcaaaggtgcgggggggttggcgcactgcgtgggttgtgcagccggccgccgcggctgtacccagcgatcaggtagcttgggctgtatcttctcaagcattaccttgtcctgggcgtaggtttgccgctagcaccatggccaccgcatccactttctcggcgttcaatgcccgctgcggcgacctgcgtcgctcggcgggctccgggccccggcgcccagcgaggcccctccccgtgcgcgggcgcgccgtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcatgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgataccttattacgtaacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagctgccttacgtgacggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgggagcagttgtcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtacatatggcttccgcggcattcaccatgtcggcgtgccccgcgatgactggcagggcccctggggcacgtcgctccggacggccagtcgccacccgcctgaggggctccaccttccagtgcctggtgaactcccacatcgacccctgcaaccagaacgtgtcctccgcctccctgtccttcctgggcgacaacggcttcggctccaaccccttccgctccaaccgcggccaccgccgcctgggccgcgcctcccactccggcgaggccatggccgtggccctgcagcccgcccaggaggtggccaccaagaagaagcccgccatcaagcagcgccgcgtggtggtgaccggcatgggcgtggtgacccccctgggccacgagcccgacgtgttctacaacaacctgctggacggcgtgtccggcatctccgagatcgagaccttcgactgcacccagttccccacccgcatcgccggcgagatcaagtccttctccaccgacggctgggtggcccccaagctgtccaagcgcatggacaagttcatgctgtacctgctgaccgccggcaagaaggccctggccgacgccggcatcaccgaggacgtgatgaaggagctggacaagcgcaagtgcggcgtgctgatcggctccggcatgggcggcatgaagctgttcaacgactccatcgaggccctgcgcgtgtcctacaagaagatgaaccccttctgcgtgcccttcgccaccaccaacatgggctccgccatgctggccatggacctgggctggatgggccccaactactccatctccaccgcctgcgccacctccaacttctgcatcctgaacgccgccaaccacatcatccgcggcgaggccgacatgatgctgtgcggcggctccgacgccgtgatcatccccatcggcctgggcggcttcgtggcctgccgcgccctgtcccagcgcaactccgaccccaccaaggcctcccgcccctgggactccaaccgcgacggcttcgtgatgggcgagggcgccggcgtgctgctgctggaggagctggagcacgccaagaagcgcggcgccaccatctacgccgagttcctgggcggctccttcacctgcgacgcctaccacatgaccgagccccaccccgacggcgccggcgtgatcctgtgcatcgagaaggccctggcccagtccggcgtgtcccgcgaggacgtgaactacatcaacgcccacgccacctccacccccgccggcgacatcaaggagtaccaggccctggcccactgcttcggccagaactccgagctgcgcgtgaactccaccaagtccatgatcggccacctgctgggcgccgccggcggcgtggaggccgtgaccgtgatccaggccatccgcaccggctggatccaccccaacctgaacctggaggaccccgacgaggccgtggacgccaagttcctggtgggccccaagaaggagcgcctgaacgtgaaggtgggcctgtccaactccttcggcttcggcggccacaactcctccatcctgttcgccccctacaacaccatgtacccctacgacgtgcccgactacgcctgagatatcggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctcagcgtctgcgtgttgggagctggagtcgtgggcttgacgacggcgctgcagctgttgcaggatgtgcctggcgtgcgcgttcacgtcgtggctgagaaatatggcgacgaaacgttgacggctggggccggcgggctgtggatgccatacgcattgggtacgcggccattggatgggattgataggcttatggagggataatagagtttttgccggatccaacgcatgtggatgcggtatcccggtgggctgaaagtgtggaaggatagtgcattggctattcacatgcactgcccaccccttttggcaggaaatgtgccggcatcgttggtgcaccgatggggaaaatcgacgttcgaccactacatgaagatttatacgtctgaagatgcagcgactgcgggtgcgaaacggatgacggtttggtcgtgtatgtcacagcatgtgctggatcttgcgggctaactccccctgccacggcccattgcaggtgtcatgttgactggagggtacgacctttcgtccgtcaaattcccagaggaggacccgctctgggccgacattgtgcccactgaagagcgtttaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagga

SEQ ID NO: 3 - protein, 532 aa, synthetic construct
  REGION 1..532: note = synthetic
MASAAFTMSACPAMTGRAPGARRSGRPVATRLRGSTFQCLVNSHIDPCNQNVSSASLSFLGDNGFGSNPFRSNRGHRRLGRASHSGEAMAVALQPAQEVATKKKPAIKQRRVVVTGMGVVTPLGHEPDVFYNNLLDGVSGISEIETFDCTQFPTRIAGEIKSFSTDGWVAPKLSKRMDKFMLYLLTAGKKALADAGITEDVMKELDKRKCGVLIGSGMGGMKLFNDSIEALRVSYKKMNPFCVPFATTNMGSAMLAMDLGWMGPNYSISTACATSNFCILNAANHIIRGEADMMLCGGSDAVIIPIGLGGFVACRALSQRNSDPTKASRPWDSNRDGFVMGEGAGVLLLEELEHAKKRGATIYAEFLGGSFTCDAYHMTEPHPDGAGVILCIEKALAQSGVSREDVNYINAHATSTPAGDIKEYQALAHCFGQNSELRVNSTKSMIGHLLGAAGGVEAVTVIQAIRTGWIHPNLNLEDPDEAVDAKFLVGPKKERLNVKVGLSNSFGFGGHNSSILFAPYNTMYPYDVPDYA

SEQ ID NO: 4 - protein, 532 aa, synthetic construct
  REGION 1..532: note = synthetic
  VARIANT 146: note = MISC_FEATURE - wherein Xaa is A,C,D,E,F,G,H,I,K,L,M,N,P,Q,R,S,V,W, or Y.
MASAAFTMSACPAMTGRAPGARRSGRPVATRLRGSTFQCLVNSHIDPCNQNVSSASLSFLGDNGFGSNPFRSNRGHRRLGRASHSGEAMAVALQPAQEVATKKKPAIKQRRVVVTGMGVVTPLGHEPDVFYNNLLDGVSGISEIEXFDCTQFPTRIAGEIKSFSTDGWVAPKLSKRMDKFMLYLLTAGKKALADAGITEDVMKELDKRKCGVLIGSGMGGMKLFNDSIEALRVSYKKMNPFCVPFATTNMGSAMLAMDLGWMGPNYSISTACATSNFCILNAANHIIRGEADMMLCGGSDAVIIPIGLGGFVACRALSQRNSDPTKASRPWDSNRDGFVMGEGAGVLLLEELEHAKKRGATIYAEFLGGSFTCDAYHMTEPHPDGAGVILCIEKALAQSGVSREDVNYINAHATSTPAGDIKEYQALAHCFGQNSELRVNSTKSMIGHLLGAAGGVEAVTVIQAIRTGWIHPNLNLEDPDEAVDAKFLVGPKKERLNVKVGLSNSFGFGGHNSSILFAPYNTMYPYDVPDYA

SEQ ID NO: 5 - protein, 400 aa, synthetic construct
  REGION 1..400: note = synthetic
MATASTFSAFNARCGDLRRSAGSGPRRPARPLPVRAAINASAHPKANGSAVNLKSGSLNTQEDTSSSPPPRAFLNQLPDWSMLVDSVGLKSVVLDGLVSRQIFSIRSYEIGADRTASIETLMNHLQETSINHCKSLGLLNDGFGRTPGMCKNDLIWVLTKMQIMVNRYPTWGDTVEINTWFSHSGKIGMASDWLITDCNTGEILIRATSVWAMMNQKTRRFSRLPYEVRQELTPHYVDSPHVIEDNDRKLHKFDVKTGDSIRKGLTPRWNDLDVNQHVSNVKYIGWILESMPIEVLETQELCSLTVEYRRECGMDSVLESVTAMDPSEDEGRSQYKHLLRLEDGTDIVKGRTEWRPKNAGTNGAISTAKPSNGNSVSMDYKDHDGDYKDHDIDYKDDDDK

SEQ ID NO: 6 - DNA, 9693 bp, synthetic construct
  misc_feature 1..9693: note = synthetic
  source 1..9693: mol_type = other DNA
agcggaagagcgcccaatgtttaaacccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggattatcaaaaacgcctgagacacttgcccaggattgaaactccctgaagggaccaccaggggccctgagttgttccttccccccgtggcgagctgccagccaggctgtacctgtgatcggggctggcgggaaaacaggcttcgtgtgctcaggttatgggaggtgcaggacagctcattaaacgccaacaatcgcacaattcatggcaagctaatcagttatttcccattaacgagctataattgtcccaaaattctggtctaccgggggtgatccttcgtgtacgggcccttccctcaaccctaggtatgcgcacatgcggtcgccgcgcaacgcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggtgtaggatttcgtaatcattcgtcctgatggggagctaccgactgccctagtatcagcccgactgcctgacgccagcgtccacttttgtgcacacattccattcgtgcccaagacatttcattgtggtgcgaagcgtccccagttacgctcacctgatccccaacctccttattgttctgtcgacagagtgggcccagaggccggtcgcagccactagtacatatggcttccgcggcattcaccatgtcggcgtgccccgcgatgactggcagggcccctggggcacgtcgctccggacggccagtcgccacccgcctgaggggctccaccttccagtgcctggtgaactcccacatcgacccctgcaaccagaacgtgtcctccgcctccctgtccttcctgggcgacaacggcttcggctccaaccccttccgctccaaccgcggccaccgccgcctgggccgcgcctcccactccggcgaggccatggccgtggccctgcagcccgcccaggaggtggccaccaagaagaagcccgccatcaagcagcgccgcgtggtggtgaccggcatgggcgtggtgaccccgctgggccacgagcccgacgtgttctacaacaacctgctggacggcgtgtccggcatctccgagatcgagagcttcgactgcacccagttccccacccgcatcgccggcgagatcaagtccttctccaccgacggctgggtggccccgaagctgtccaagcgcatggacaagttcatgctgtacctgctgaccgccggcaagaaggccctggccgacgccggcatcaccgaggacgtgatgaaggagctggacaagcgcaagtgcggcgtgctgatcggctccggcatgggcggcatgaagctgttcaacgactccatcgaggccctgcgcgtgtcctacaagaagatgaaccccttctgcgtgcccttcgccaccaccaacatgggctccgccatgctggccatggacctgggctggatgggccccaactactccatctccaccgcctgcgccacctccaacttctgcatcctgaacgccgccaaccacatcatccgcggcgaggccgacatgatgctgtgcggcggctccgacgccgtgatcatccccatcggcctgggcggcttcgtggcctgccgcgccctgtcccagcgcaactccgaccccaccaaggcctcccgcccctgggactccaaccgcgacggcttcgtgatgggcgagggcgccggcgtgctgctgctggaggagctggagcacgccaagaagcgcggcgccaccatctacgccgagttcctgggcggctccttcacctgcgacgcctaccacatgaccgagccgcacccggacggcgccggcgtgatcctgtgcatcgagaaggccctggcccagtccggcgtgtcccgcgaggacgtgaactacatcaacgcccacgccacctccaccccggccggcgacatcaaggagtaccaggccctggcccactgcttcggccagaactccgagctgcgcgtgaactccaccaagtccatgatcggccacctgctgggcgccgccggcggcgtggaggccgtgaccgtgatccaggccatccgcaccggctggatccaccccaacctgaacctggaggaccccgacgaggccgtggacgccaagttcctggtgggccccaagaaggagcgcctgaacgtgaaggtgggcctgtccaactccttcggcttcggcggccacaactcctccatcctgttcgccccgtacaacaccatgtacccctacgacgtgcccgactacgcctgagatatcggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgactgaagagcgtttaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggcaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagga

SEQ ID NO: 7 - DNA, 1599 bp, synthetic construct
  misc_feature 1..1599: note = synthetic
  source 1..1599: mol_type = other DNA
atggcttccgcggcattcaccatgtcggcgtgccccgcgatgactggcagggcccctggggcacgtcgctccggacggccagtcgccacccgcctgaggggctccaccttccagtgcctggtgaactcccacatcgacccctgcaaccagaacgtgtcctccgcctccctgtccttcctgggcgacaacggcttcggctccaaccccttccgctccaaccgcggccaccgccgcctgggccgcgcctcccactccggcgaggccatggccgtggccctgcagcccgcccaggaggtggccaccaagaagaagcccgccatcaagcagcgccgcgtggtggtgaccggcatgggcgtggtgaccccgctgggccacgagcccgacgtgttctacaacaacctgctggacggcgtgtccggcatctccgagatcgagaccttcgactgcacccagttccccacccgcatcgccggcgagatcaagtccttctccaccgacggctgggtggccccgaagctgtccaagcgcatggacaagttcatgctgtacctgctgaccgccggcaagaaggccctggccgacgccggcatcaccgaggacgtgatgaaggagctggacaagcgcaagtgcggcgtgctgatcggctccggcatgggcggcatgaagctgttcaacgactccatcgaggccctgcgcgtgtcctacaagaagatgaaccccttctgcgtgcccttcgccaccaccaacatgggctccgccatgctggccatggacctgggctggatgggccccaactactccatctccaccgcctgcgccacctccaacttctgcatcctgaacgccgccaaccacatcatccgcggcgaggccgacatgatgctgtgcggcggctccgacgccgtgatcatccccatcggcctgggcggcttcgtggcctgccgcgccctgtcccagcgcaactccgaccccaccaaggcctcccgcccctgggactccaaccgcgacggcttcgtgatgggcgagggcgccggcgtgctgctgctggaggagctggagcacgccaagaagcgcggcgccaccatctacgccgagttcctgggcggctccttcacctgcgacgcctaccacatgaccgagccgcacccggacggcgccggcgtgatcctgtgcatcgagaaggccctggcccagtccggcgtgtcccgcgaggacgtgaactacatcaacgcccacgccacctccaccccggccggcgacatcaaggagtaccaggccctggcccactgcttcggccagaactccgagctgcgcgtgaactccaccaagtccatgatcggccacctgctgggcgccgccggcggcgtggaggccgtgaccgtgatccaggccatccgcaccggctggatccaccccaacctgaacctggaggaccccgacgaggccgtggacgccaagttcctggtgggccccaagaaggagcgcctgaacgtgaaggtgggcctgtccaactccttcggcttcggcggccacaactcctccatcctgttcgccccgtacaacaccatgtacccctacgacgtgcccgactacgcctga

SEQ ID NO: 8 - DNA, 9520 bp, synthetic construct
  misc_feature 1..9520: note = synthetic
  source 1..9520: mol_type = other DNA
gatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatgtttaaacagcccgcaccctcgttgatctgggagccctgcgcagccccttaaatcatctcagtcaggtttctgtgttcaactgagcctaaagggctttcgtcatgcgcacgagcacacgtatatcggccacgcagtttctcaaaagcggtagaacagttcgcgagccctcgtaggtcgaaaacttgcgccagtactattaaattaaattaattgatcgaacgagacgcgaaacttttgcagaatgccaccgagtttgcccagagaatgggagtggcgccattcaccatccgcctgtgcccggcttgattcgccgagacgatggacggcgagaccagggagcggcttgcgagccccgagccggtagcaggaacaatgatcgacaatcttcctgtccaattactggcaaccattagaaagagccggagcgcgttgaaagtctgcaatcgagtaatttttcgatacgtcgggcctgctgaaccctaaggctccggactttgtttaaggcgatccaagatgcacgcggccccaggcacgtatctcaagcacaaaccccagccttagtttcgagactttgggagatagcgaccgatatctagtttggcattttgtatattaattacctcaagcaatggagcgctctgatgcggtgcagcgtcggctgcagcacctggcagtggcgctagggtcgccctatcgctcggaacctggtcagctggctcccgcctcctgctcagcctcttccggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcagctagcaccatggccaccgcatccactttctcggcgttcaatgcccgctgcggcgacctgcgtcgctcggcgggctccgggccccggcgcccagcgaggcccctccccgtgcgcgggcgcgcccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcatgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgataccttattacgtaacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagctgccttacgtgacggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcatcaaaaacgcctgagacacttgcccaggattgaaactccctgaagggaccaccaggggccctgagttgttccttccccccgtggcgagctgccagccaggctgtacctgtgatcggggctggcgggaaaacaggcttcgtgtgctcaggttatgggaggtgcaggacagctcattaaacgccaacaatcgcacaattcatggcaagctaatcagttatttcccattaacgagctataattgtcccaaaattctggtctaccgggggtgatccttcgtgtacgggcccttccctcaaccctaggtatgcgcacatgcggtcgccgcgcaacgcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggtgtaggatttcgtaatcattcgtcctgatggggagctaccgactgccctagtatcagcccgactgcctgacgccagcgtccacttttgtgcacacattccattcgtgcccaagacatttcattgtggtgcgaagcgtccccagttacgctcacctgatccccaacctccttattgttctgtcgacagagtgggcccagaggccggtcgcagccactagtaacaatggccaccgcctccaccttctccgccttcaacgcccgctgcggcgacctgcgccgctccgccggctccggcccccgccgccccgcccgccccctgcccgtgcgcgccgccatcaacgcctccgcccaccccaaggccaacggctccgccgtgaacctgaagtccggctccctgaacacccaggaggacacctcctcctctccgcctccccgcgccttcctgaaccagctgcccgactggtccatgctggtggactccgtgggcctgaagtccgtggtgctggacggcctggtgtcccgccagatcttctccatccgctcctacgagatcggcgccgaccgcaccgcctccatcgagaccctgatgaaccacctgcaggagacctccatcaaccactgcaagtccctgggcctgctgaacgacggcttcggccgcacccccggcatgtgcaagaacgacctgatctgggtgctgaccaagatgcagatcatggtgaaccgctaccccacctggggcgacaccgtggagatcaacacctggttctcccactccggcaagatcggcatggcctccgactggctgatcaccgactgcaacaccggcgagatcctgatccgcgccacctccgtgtgggccatgatgaaccagaagacccgccgcttctcccgcctgccctacgaggtgcgccaggagctgacccctcactacgtggactccccgcacgtgatcgaggacaacgaccgcaagctgcacaagttcgacgtgaagaccggcgactccatccgcaagggcctgacccctcgctggaacgacctggacgtgaaccagcacgtgtccaacgtgaagtacatcggctggatcctggagtccatgcccatcgaggtgctggagacccaggagctgtgctccctgaccgtggagtaccgccgcgagtgcggcatggactccgtgctggagtccgtgaccgccatggacccctccgaggacgagggccgctcccagtacaagcacctgctgcgcctggaggacggcaccgacatcgtgaagggccgcaccgagtggcgccccaagaacgccggcaccaacggcgccatctccaccgccaagccctccaacggcaactccgtgtccatggactacaaggaccacgacggcgactacaaggaccacgacatcgactacaaggacgacgacgacaagtgactcgagggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctcagcgtctgcgtgttgggagctggagtcgtgggcttgacgacggcgctgcagctgttgcaggatgtgcctggcgtgcgcgttcacgtcgtggctgagaaatatggcgacgaaacgttgacggctggggccggcgggctgtggatgccatacgcattgggtacgcggccattggatgggattgataggcttatggagggataatagagtttttgccggatccaacgcatgtggatgcggtatcccggtgggctgaaagtgtggaaggatagtgcattggctattcacatgcactgcccaccccttttggcaggaaatgtgccggcatcgttggtgcaccgatggggaaaatcgacgttcgaccactacatgaagatttatacgtctgaagatgcagcgactgcgggtgcgaaacggatgacggtttggtcgtgtatgtcacagcatgtgctggatcttgcgggctaactccccctgccacggcccattgcaggtgtcatgttgactggagggtacgacctttcgtccgtcaaattcccagaggaggacccgctctgggccgacattgtgcccactgaagagcgtttaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaag

SEQ ID NO: 9 - DNA, 1599 bp, synthetic construct
  misc_feature 1..1599: note = synthetic
  source 1..1599: mol_type = other DNA
atggcttccgcggcattcaccatgtcggcgtgccccgcgatgactggcagggcccctggggcacgtcgctccggacggccagtcgccacccgcctgaggggctccaccttccagtgcctggtgaactcccacatcgacccctgcaaccagaacgtgtcctccgcctccctgtccttcctgggcgacaacggcttcggctccaaccccttccgctccaaccgcggccaccgccgcctgggccgcgcctcccactccggcgaggccatggccgtggccctgcagcccgcccaggaggtggccaccaagaagaagcccgccatcaagcagcgccgcgtggtggtgaccggcatgggcgtggtgaccccgctgggccacgagcccgacgtgttctacaacaacctgctggacggcgtgtccggcatctccgagatcgagagcttcgactgcacccagttccccacccgcatcgccggcgagatcaagtccttctccaccgacggctgggtggccccgaagctgtccaagcgcatggacaagttcatgctgtacctgctgaccgccggcaagaaggccctggccgacgccggcatcaccgaggacgtgatgaaggagctggacaagcgcaagtgcggcgtgctgatcggctccggcatgggcggcatgaagctgttcaacgactccatcgaggccctgcgcgtgtcctacaagaagatgaaccccttctgcgtgcccttcgccaccaccaacatgggctccgccatgctggccatggacctgggctggatgggccccaactactccatctccaccgcctgcgccacctccaacttctgcatcctgaacgccgccaaccacatcatccgcggcgaggccgacatgatgctgtgcggcggctccgacgccgtgatcatccccatcggcctgggcggcttcgtggcctgccgcgccctgtcccagcgcaactccgaccccaccaaggcctcccgcccctgggactccaaccgcgacggcttcgtgatgggcgagggcgccggcgtgctgctgctggaggagctggagcacgccaagaagcgcggcgccaccatctacgccgagttcctgggcggctccttcacctgcgacgcctaccacatgaccgagccgcacccggacggcgccggcgtgatcctgtgcatcgagaaggccctggcccagtccggcgtgtcccgcgaggacgtgaactacatcaacgcccacgccacctccaccccggccggcgacatcaaggagtaccaggccctggcccactgcttcggccagaactccgagctgcgcgtgaactccaccaagtccatgatcggccacctgctgggcgccgccggcggcgtggaggccgtgaccgtgatccaggccatccgcaccggctggatccaccccaacctgaacctggaggaccccgacgaggccgtggacgccaagttcctggtgggccccaagaaggagcgcctgaacgtgaaggtgggcctgtccaactccttcggcttcggcggccacaactcctccatcctgttcgccccgtacaacaccatgtacccctacgacgtgcccgactacgcctga

SEQ ID NO: 10 - protein, 38 aa, synthetic construct
  REGION 1..38: note = synthetic
MATASTFSAFNARCGDLRRSAGSGPRRPARPLPVRGRA

SEQ ID NO: 11 - protein, 38 aa, synthetic construct
  REGION 1..38: note = synthetic
MATASTFSAFNARCGDLRRSAGSGPRRPARPLPVRAAI

SEQ ID NO: 12 - DNA, 114 bp, synthetic construct
  misc_feature 1..114: note = synthetic
  source 1..114: mol_type = other DNA
atggccaccgcctccaccttctccgccttcaacgcccgctgcggcgacctgcgccgctccgccggctccggcccccgccgccccgcccgccccctgcccgtgcgcgccgccatc

SEQ ID NO: 13 - DNA, 9574 bp, synthetic construct
  misc_feature 1..9574: note = synthetic
  source 1..9574: mol_type = other DNA
agcggaagagcgcccaatgtttaaacccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatgggagcagttgtcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtacatatggcttccgcggcattcaccatgtcggcgtgccccgcgatgactggcagggcccctggggcacgtcgctccggacggccagtcgccacccgcctgaggggctccaccttccagtgcctggtgaactcccacatcgacccctgcaaccagaacgtgtcctccgcctccctgtccttcctgggcgacaacggcttcggctccaaccccttccgctccaaccgcggccaccgccgcctgggccgcgcctcccactccggcgaggccatggccgtggccctgcagcccgcccaggaggtggccaccaagaagaagcccgccatcaagcagcgccgcgtggtggtgaccggcatgggcgtggtgaccccgctgggccacgagcccgacgtgttctacaacaacctgctggacggcgtgtccggcatctccgagatcgagaccttcgactgcacccagttccccacccgcatcgccggcgagatcaagtccttctccaccgacggctgggtggccccgaagctgtccaagcgcatggacaagttcatgctgtacctgctgaccgccggcaagaaggccctggccgacgccggcatcaccgaggacgtgatgaaggagctggacaagcgcaagtgcggcgtgctgatcggctccggcatgggcggcatgaagctgttcaacgactccatcgaggccctgcgcgtgtcctacaagaagatgaaccccttctgcgtgcccttcgccaccaccaacatgggctccgccatgctggccatggacctgggctggatgggccccaactactccatctccaccgcctgcgccacctccaacttctgcatcctgaacgccgccaaccacatcatccgcggcgaggccgacatgatgctgtgcggcggctccgacgccgtgatcatccccatcggcctgggcggcttcgtggcctgccgcgccctgtcccagcgcaactccgaccccaccaaggcctcccgcccctgggactccaaccgcgacggcttcgtgatgggcgagggcgccggcgtgctgctgctggaggagctggagcacgccaagaagcgcggcgccaccatctacgccgagttcctgggcggctccttcacctgcgacgcctaccacatgaccgagccgcacccggacggcgccggcgtgatcctgtgcatcgagaaggccctggcccagtccggcgtgtcccgcgaggacgtgaactacatcaacgcccacgccacctccaccccggccggcgacatcaaggagtaccaggccctggcccactgcttcggccagaactccgagctgcgcgtgaactccaccaagtccatgatcggccacctgctgggcgccgccggcggcgtggaggccgtgaccgtgatccaggccatccgcaccggctggatccaccccaacctgaacctggaggaccccgacgaggccgtggacgccaagttcctggtgggccccaagaaggagcgcctgaacgtgaaggtgggcctgtccaactccttcggcttcggcggccacaactcctccatcctgttcgccccgtacaacaccatgtacccctacgacgtgcccgactacgcctgagatatcggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgactgaagagcgtttaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggcaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagga

SEQ ID NO: 14 - DNA, 1599 bp, synthetic construct
  misc_feature 1..1599: note = synthetic
  misc_difference 436..438: note = n is, independently, a, c, g, or t
  source 1..1599: mol_type = other DNA
atggcttccgcggcattcaccatgtcggcgtgccccgcgatgactggcagggcccctggggcacgtcgctccggacggccagtcgccacccgcctgaggggctccaccttccagtgcctggtgaactcccacatcgacccctgcaaccagaacgtgtcctccgcctccctgtccttcctgggcgacaacggcttcggctccaaccccttccgctccaaccgcggccaccgccgcctgggccgcgcctcccactccggcgaggccatggccgtggccctgcagcccgcccaggaggtggccaccaagaagaagcccgccatcaagcagcgccgcgtggtggtgaccggcatgggcgtggtgaccccgctgggccacgagcccgacgtgttctacaacaacctgctggacggcgtgtccggcatctccgagatcgagnnnttcgactgcacccagttccccacccgcatcgccggcgagatcaagtccttctccaccgacggctgggtggccccgaagctgtccaagcgcatggacaagttcatgctgtacctgctgaccgccggcaagaaggccctggccgacgccggcatcaccgaggacgtgatgaaggagctggacaagcgcaagtgcggcgtgctgatcggctccggcatgggcggcatgaagctgttcaacgactccatcgaggccctgcgcgtgtcctacaagaagatgaaccccttctgcgtgcccttcgccaccaccaacatgggctccgccatgctggccatggacctgggctggatgggccccaactactccatctccaccgcctgcgccacctccaacttctgcatcctgaacgccgccaaccacatcatccgcggcgaggccgacatgatgctgtgcggcggctccgacgccgtgatcatccccatcggcctgggcggcttcgtggcctgccgcgccctgtcccagcgcaactccgaccccaccaaggcctcccgcccctgggactccaaccgcgacggcttcgtgatgggcgagggcgccggcgtgctgctgctggaggagctggagcacgccaagaagcgcggcgccaccatctacgccgagttcctgggcggctccttcacctgcgacgcctaccacatgaccgagccgcacccggacggcgccggcgtgatcctgtgcatcgagaaggccctggcccagtccggcgtgtcccgcgaggacgtgaactacatcaacgcccacgccacctccaccccggccggcgacatcaaggagtaccaggccctggcccactgcttcggccagaactccgagctgcgcgtgaactccaccaagtccatgatcggccacctgctgggcgccgccggcggcgtggaggccgtgaccgtgatccaggccatccgcaccggctggatccaccccaacctgaacctggaggaccccgacgaggccgtggacgccaagttcctggtgggccccaagaaggagcgcctgaacgtgaaggtgggcctgtccaactccttcggcttcggcggccacaactcctccatcctgttcgccccgtacaacaccatgtacccctacgacgtgcccgactacgcctga

SEQ ID NO: 15 - DNA, 9402 bp, synthetic construct
  misc_feature 1..9402: note = synthetic
  source 1..9402: mol_type = other DNA
gatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatgtttaaacagcccgcaccctcgttgatctgggagccctgcgcagccccttaaatcatctcagtcaggtttctgtgttcaactgagcctaaagggctttcgtcatgcgcacgagcacacgtatatcggccacgcagtttctcaaaagcggtagaacagttcgcgagccctcgtaggtcgaaaacttgcgccagtactattaaattaaattaattgatcgaacgagacgcgaaacttttgcagaatgccaccgagtttgcccagagaatgggagtggcgccattcaccatccgcctgtgcccggcttgattcgccgagacgatggacggcgagaccagggagcggcttgcgagccccgagccggtagcaggaacaatgatcgacaatcttcctgtccaattactggcaaccattagaaagagccggagcgcgttgaaagtctgcaatcgagtaatttttcgatacgtcgggcctgctgaaccctaaggctccggactttgtttaaggcgatccaagatgcacgcggccccaggcacgtatctcaagcacaaaccccagccttagtttcgagactttgggagatagcgaccgatatctagtttggcattttgtatattaattacctcaagcaatggagcgctctgatgcggtgcagcgtcggctgcagcacctggcagtggcgctagggtcgccctatcgctcggaacctggtcagctggctcccgcctcctgctcagcctcttccggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcagctagcaccatggccaccgcatccactttctcggcgttcaatgcccgctgcggcgacctgcgtcgctcggcgggctccgggccccggcgcccagcgaggcccctccccgtgcgcgggcgcgcccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcatgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgataccttattacgtaacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagctgccttacgtgacggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgggagcagttgtcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtaacaatggccaccgcctccaccttctccgccttcaacgcccgctgcggcgacctgcgccgctccgccggctccggcccccgccgccccgcccgccccctgcccgtgcgcgccgccatcaacgcctccgcccaccccaaggccaacggctccgccgtgaacctgaagtccggctccctgaacacccaggaggacacctcctcctctccgcctccccgcgccttcctgaaccagctgcccgactggtccatgctggtggactccgtgggcctgaagtccgtggtgctggacggcctggtgtcccgccagatcttctccatccgctcctacgagatcggcgccgaccgcaccgcctccatcgagaccctgatgaaccacctgcaggagacctccatcaaccactgcaagtccctgggcctgctgaacgacggcttcggccgcacccccggcatgtgcaagaacgacctgatctgggtgctgaccaagatgcagatcatggtgaaccgctaccccacctggggcgacaccgtggagatcaacacctggttctcccactccggcaagatcggcatggcctccgactggctgatcaccgactgcaacaccggcgagatcctgatccgcgccacctccgtgtgggccatgatgaaccagaagacccgccgcttctcccgcctgccctacgaggtgcgccaggagctgacccctcactacgtggactccccgcacgtgatcgaggacaacgaccgcaagctgcacaagttcgacgtgaagaccggcgactccatccgcaagggcctgacccctcgctggaacgacctggacgtgaaccagcacgtgtccaacgtgaagtacatcggctggatcctggagtccatgcccatcgaggtgctggagacccaggagctgtgctccctgaccgtggagtaccgccgcgagtgcggcatggactccgtgctggagtccgtgaccgccatggacccctccgaggacgagggccgctcccagtacaagcacctgctgcgcctggaggacggcaccgacatcgtgaagggccgcaccgagtggcgccccaagaacgccggcaccaacggcgccatctccaccgccaagccctccaacggcaactccgtgtccatggactacaaggaccacgacggcgactacaaggaccacgacatcgactacaaggacgacgacgacaagtgactcgagggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctcagcgtctgcgtgttgggagctggagtcgtgggcttgacgacggcgctgcagctgttgcaggatgtgcctggcgtgcgcgttcacgtcgtggctgagaaatatggcgacgaaacgttgacggctggggccggcgggctgtggatgccatacgcattgggtacgcggccattggatgggattgataggcttatggagggataatagagtttttgccggatccaacgcatgtggatgcggtatcccggtgggctgaaagtgtggaaggatagtgcattggctattcacatgcactgcccaccccttttggcaggaaatgtgccggcatcgttggtgcaccgatggggaaaatcgacgttcgaccactacatgaagatttatacgtctgaagatgcagcgactgcgggtgcgaaacggatgacggtttggtcgtgtatgtcacagcatgtgctggatcttgcgggctaactccccctgccacggcccattgcaggtgtcatgttgactggagggtacgacctttcgtccgtcaaattcccagaggaggacccgctctgggccgacattgtgcccactgaagagcgtttaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaag

SEQ ID NO: 16 - protein, 13 aa, synthetic construct
  REGION 1..13: note = synthetic
SGPRRPARPLPVR

SEQ ID NO: 17 - protein, 28 aa, synthetic construct
  REGION 1..28: note = synthetic
SGPRRPARPLPVRAAIASEVPVATTSPR

SEQ ID NO: 18 - protein, 12 aa, synthetic construct
  REGION 1..12: note = synthetic
RPARPLPVRGRA

SEQ ID NO: 19 - protein, 24 aa, synthetic construct
  REGION 1..24: note = synthetic
RPARPLPVRAAIASEVPVATTSPR

SEQ ID NO: 20 - protein, 26 aa, synthetic construct
  REGION 1..26: note = synthetic
RCGDLRRSAGSGPRRPARPLPVRGRA

SEQ ID NO: 21 - protein, 38 aa, synthetic construct
  REGION 1..38: note = synthetic
RCGDLRRSAGSGPRRPARPLPVRAAIASEVPVATTSPR

SEQ ID NO: 22 - protein, 8 aa, synthetic construct
  REGION 1..8: note = synthetic
PARPLPVR

SEQ ID NO: 23 - protein, 23 aa, synthetic construct
  REGION 1..23: note = synthetic
PARPLPVRAAIASEVPVATTSPR

SEQ ID NO: 24 - protein, 10 aa, synthetic construct
  REGION 1..10: note = synthetic
RRPARPLPVR

SEQ ID NO: 25 - protein, 25 aa, synthetic construct
  REGION 1..25: note = synthetic
RRPARPLPVRAAIASEVPVATTSPR